(19) United States
(12) Patent Application Publication
(10) Pub. No.: US 2026/0103763 A1
(43) Pub. Date: Apr. 16, 2026

(54) LINKED TARGET CAPTURE AND PROXIMITY SEQUENCING WITH PHASED VARIANTS

(71) Applicant: NCAN Genomics, Inc., Vancouver (CA)

(72) Inventor: Joel Pel, Vancouver (CA)

(21) Appl. No.: 19/354,525

(22) Filed: Oct. 9, 2025

Related U.S. Application Data
(60) Provisional application No. 63/706,859, filed on Oct. 14, 2024.

(51) Int. Cl. C12Q 1/6886; C12Q 1/6855; C12Q 1/6869

(52) U.S. Cl. CPC C12Q 1/6886; C12Q 1/6855; C12Q 1/6869; C12Q 2600/156

(57) ABSTRACT

The invention uses (i) proximity sequencing (Pro-Seq), in which a fragment is copied redundantly in a manner such that an error introduced during copying does not appear in all copies, and/or (ii) linked target capture (LTC), whereby a nucleic acid is amplified by primers that are linked to target-specific probes to discover and/or detect phased variants as biomarkers.

### BACKGROUND OF THE INVENTION

Information from the sequences of nucleic acids has many applications in research and medicine. For example, some medical conditions may be characterized by specific mutations in genomic DNA. One common feature of cancer is that tumor DNA will have a mutation that is specific to the tumor relative to non-tumor DNA. Such a mutation may contribute to the proliferation of the tumor cells. For example, a tumor mutation may “knock out” a tumor suppressor gene and such causative mutations are sometimes known as driver mutations. Tumor genomes may also gain mutations that have no apparent contribution to the disease but are nevertheless specific to the tumor DNA and those are sometimes referred to as passenger mutations. Attempts have been made to sequence DNA from tumors and to identify a mutation that is specific to the tumor with the idea being that such a tumor-specific mutation may be used as a “biomarker” that, if detected later, is evidence of the presence of the tumor.

In many cases, a tumor-specific mutation is present as a single-nucleotide polymorphism (SNP), wherein one single base from an individual's germline DNA has been changed in a somatic cell to a different nucleotide base. It is unfortunate that a tumor-specific mutation may simply be a SNP because laboratory techniques for extracting and analyzing DNA are known to be subject to errors that often change one base to another, sometimes referred to as a sample preparation artefact, with the result that sequencing tumor DNA often shows an apparent change without the possibility to distinguish between a true SNP and a sample preparation artefact.

### SUMMARY OF THE INVENTION

The invention provides sample preparation techniques that correct for sample preparation artefacts and discover true mutations for use as biomarkers that themselves will not appear as artefacts. Preferred biomarkers of the invention are “phased variants”, which include two or more mutations (relative to a reference) that appear within a limited distance of each other on one strand of DNA from a sample. The distance may be limited to less than about 200 bases, e.g., less than about 170 bases, so that that a phased variant biomarker is one that may be found among cell-free nucleic acid (which may have a mean or modal fragment length of about 170 bases). When a sample nucleic acid is analyzed to detect a phased variant that is present in a strand of the sample nucleic acid, it is highly improbable that a sample preparation artefacts such as polymerase error will generate an artefact that matches the phased variant. Additionally, methods of the invention use sample preparation techniques that are robust against false positives due to artefacts. The nucleic acid to be analyzed may be analyzed using (i) proximity sequencing (Pro-Seq), in which a fragment is copied redundantly using linked primers to make linked copies and in a manner such that an error introduced during copying does not appear in all copies, and/or (ii) linked target capture (LTC), whereby a nucleic acid is amplified by primers that are linked to target-specific probes to provide highly target-specific enrichment, providing for redundant copes of the target and highly sensitive detection.

Methods of the invention have particular applications in cancer detection and treatment. For example, a tumor sample may be assayed to discover a phased variant that is specific to the tumor. Inherent to Pro-Seq is that true phased variants from the tumor genome will reliably be found across all sequence reads from the sample, while an artefact such as a polymerase error will only appear in a measurable proportion of the sequence reads and may be identified as an artefact. Phased variants are a powerful target type because the likelihood of having two polymerase errors that match a phased variant is very low. Not only do methods of the invention have utility in discovering phased variant biomarkers that are specific to tumors, methods of the invention are particularly well-suited for the detection of such a biomarker, after it has been discovered, later in a patient's journey. For example, after a patient has been treated to remove a tumor, it may be clinically important to later detect any evidence of the tumor in the patient. That is, it may be important to detect any minimal residual disease (MRD). Sample preparation methods of the invention are well suited for the detection of a phased variant biomarker as evidence of MRD due to their specificity and robustness. For example, LTC may be used to specifically enrich for a DNA fragment that harbors a phased variant biomarker, even when than fragment is present only in small quantities, such as a single fragment in liquid biopsy sample such as a 10 to 50 mL blood draw.

As shown herein, Pro-Seq and LTC have particular benefits in discovering and detecting phased variants as biomarkers and thus may be used in the detection and management of medical conditions such as cancer.

In certain aspects, the invention provides a method of using linked target capture (LTC) to detect minimal residual disease (MRD). The method includes providing a set of probe-dependent primers (PDPs) in which each PDP includes a universal primer linked to a probe designed to anneal to a segment of DNA harboring first and second variants that are (i) specific to a tumor of a subject, and (ii) were found present together on one strand of nucleic acid from the tumor. PDPs of the set enrich either for fragments with the variants or genomic regions where the variants are expected. Methods include ligating an adaptor comprising a universal priming site to a fragment from a sample from the subject; hybridizing probes of the PDP set to the fragment and hybridizing the universal primer to the universal priming site; extending the universal primer with a strand-displacing polymerase to make a copy of the fragment; sequencing the copy of the fragment to generate sequence data; analyzing the sequence data to detect the first and second variants on the fragment to thereby indicate the presence of the tumor in the subject. The first and second variants may have been previously found as phased variants on one strand of the segment of DNA from the tumor. The subject may have been treated to remove the tumor. The sample from the subject may comprises blood or plasma and the fragment from the sample may be cell-free DNA (cfDNA) in the blood or plasma. The adaptors may include barcodes such unique molecular identifiers (UMIs) or optionally UMIs may be added at any other stage of the workflow, such as from the PDPs. Including UMIs may be used to aid in identifying and/or quantifying variants in a sample.

In some embodiments, the copy of the fragment includes copies of a universal priming site at both ends (e.g., from ligated adaptors) and the method includes amplifying the copy of the fragment to generate amplicons prior to the sequencing step, and the sequencing step includes sequencing the amplicons.

Methods may include, prior to the providing step, performing a phased variant biomarker discovery process using LTC to discover and select the phased variant biomarker for the tumor. Discovery by LTC may include: ligating an adaptor comprising a universal priming site to a tumor nucleic acid from a tumor sample from the subject to produce an adaptor-ligated fragment; exposing the adaptor-ligated fragment to a universal primer linked to a probe (a PDP) and hybridizing the probe to a site within the tumor nucleic acid and hybridizing the universal primer to the universal priming site; extending the universal primer with a strand-displacing polymerase to make a copy of the fragment; sequencing the copy to identify, by comparing to non-tumor genomic data, the first and second variants as a tumor-specific, phased variant biomarker.

Methods may include, prior to the providing step, using Pro-Seq to discover the first and second variants as a tumor-specific, phased variant biomarker. Discovery by Pro-Seq may include exposing tumor nucleic acid from a tumor sample from the subject to a first primer that (i) anneals to the tumor nucleic acid, and (ii) is linked to a second primer than anneals to the tumor nucleic acid; extending the first primer and the second primer to produce a complex comprising linked first and second copies of the tumor nucleic acid in which the linked first and second copies have substantially the same sequence; generating sequences from the linked first and second copies; comparing the sequences to non-tumor genomic data to identify the first and second variants as a tumor-specific, phased variant biomarker. Discovery by Pro-Seq may include diluting and partitioning the tumor sample into aqueous partitions (e.g., droplets or wells in a plate) to isolate the tumor nucleic acid in one partition and performing the exposing step in the one partition. Discovery by Pro-Seq may include generating the sequences by capturing the linked first and second copies to oligos attached to a solid support; performing an amplification reaction that includes extending the oligos to generate amplicons that are all: direct or indirect copies of the linked first and second copies, and attached to the solid support as a cluster; conducting a sequencing reaction on the amplicons to generate signal for positions within the amplicons; and determining the sequences as the consensus among the signal for the positions within the amplicons.

In some embodiments, the solid support is a bead and the sequencing reaction is conducted on the cluster while the bead is held in a position on a substrate or flow cell. The solid support may be a flow cell and the amplification may be bridge amplification.

In certain embodiments, the first and second variants are each a single-nucleotide polymorphism that were previously found present together as phased variants fewer than two hundred bases apart (e.g., less than about 170 bases, or 100, or 90, etc.) on the one strand of the nucleic acid from the tumor. Non-tumor nucleic acid from the subject, homologous to the one strand of nucleic acid from the tumor, may be sequenced and shown to not contain the first or the second variant. Methods may include obtaining samples from the subject at a plurality of different times after treatment to remove the tumor and sequencing cell-free DNA from the samples to detect the phased variants as evidence of MRD.

Aspects of the invention provide method of using proximity sequencing (Pro-Seq) to detect MRD. The method includes: isolating a nucleic acid fragment from a sample from a subject in a reaction volume (e.g., droplet or well) with a first primer and a second primer that is linked to the first primer; extending the first primer and the second primer to produce a complex comprising first and second copies of the fragment linked together at their 5′ ends wherein the first copy of the fragment and the second copy of the fragment have substantially the same sequence; conducting an amplification reaction with the complex to create a cluster of amplicons copied from the first copy of the fragment and the second copy of the fragment; sequencing the cluster to generate sequence reads; assigning a base identity to each position in the fragment for which the sequence reads are in consensus thereby providing a base sequence of the fragment; and analyzing the base sequence of the fragment to detect first and second variants that were found to be specific to a tumor of the subject when found present together on one strand of nucleic acid from the tumor.

The first copy of the fragment may be synthesized by a polymerase copying a first strand of the fragment and the second copy of the fragment may be synthesized by a polymerase copying a copy of a second strand of the fragment, complementary to the first strand.

In some embodiments, the first primer is linked to the second primer by both being linked to a bead. The bead may have a plurality of primers and the method may include extending the plurality of primers to create the cluster of amplicons all linked to the bead. In certain embodiments, the cluster of amplicons linked to the bead include a first plurality of copies derived from a first strand of the fragment and a second plurality of copies derived from a second strand of the fragment. The cluster on the bead may be sequenced, e.g., within a well or on a flow cell. Because the first copy from the fragment and the second copy from the fragment may be generated by copying to two different sense strands, respectively, of a double stranded fragment, sequencing the cluster sequences copies from both senses simultaneously.

Preferably copies derived from a first strand and the copies derived from a second strand (i.e., copies from both strands of a duplex, e.g., a sense strand and its complementary antisense strand) are both present in the cluster in substantial quantities. In some embodiments, the copies derived from a first strand are attached to a first barcode and the copies derived from a second strand are attached to a second barcode. Methods may include analyzing the sequence reads to confirm the presence of the first barcode and the second barcode to confirm that the copies derived from a first strand and the copies derived from a second strand were both sequenced.

The isolating step may include diluting the sample so that the nucleic acid fragment is isolated in the reaction volume, e.g., droplet or well.

Methods may include, prior to detection using Pro-Seq, using Pro-Seq to discover the phased variant biomarker by: exposing tumor nucleic acid from a tumor sample from the subject to a first primer that (i) anneals to the tumor nucleic acid, and (ii) is linked to a second primer than anneals to the tumor nucleic acid; extending the first primer and the second primer to produce a complex comprising linked first and second copies of the tumor nucleic acid; generating sequences from the linked first and second copies; comparing the sequences to non-tumor genomic data to identify the first and second variants as a tumor-specific, phased variant biomarker.

Methods may include, prior to detection using Pro-Seq, using linked target capture (LTC) to discover the phased variant biomarker by: ligating an adaptor comprises a universal priming site to tumor nucleic acid from a tumor sample from the subject to produce an adaptor-ligated fragment (adaptors are preferably ligated at both ends of fragments); capturing the adaptor-ligated fragment with a universal primer linked to a probe by hybridizing the probe to a site within the tumor nucleic acid and hybridizing the universal primer to the universal priming site; extending the universal primer with a strand-displacing polymerase to make a copy of the fragment; sequencing the copy to identify, by comparing to non-tumor genomic data, the first and second variants as a tumor-specific, phased variant biomarker.

Other aspects of the invention provide a method of using proximity sequencing (Pro-Seq) for discovering or selecting a cancer biomarker. The method includes: exposing a nucleic acid fragment, or copies thereof, from a tumor from a subject to a first primer and a second primer that is linked to the first primer; extending the first primer and the second primer to produce a complex comprising a first copy of the fragment and a second copy of the fragment linked together at their 5′ ends wherein the first copy of the fragment and the second copy of the fragment have substantially the same sequence; generating sequence data from the complex; comparing the sequence data to non-tumor genomic data to identify a first variant and a second variant, relative to the non-tumor genomic data, present on the nucleic acid from the tumor as phased variants for use as a phased variant biomarker specific to the tumor. The first copy of the fragment may be synthesized by a polymerase copying a first strand of the fragment and the second copy of the fragment may be synthesized by a polymerase copying a copy of a second strand of the fragment, complementary to the first strand. In some embodiments, the first primer is linked to the second primer by both being linked to a bead, e.g., a bead linked to a plurality of primers and the method may include extending the plurality of primers to create the cluster of amplicons all linked to the bead. The cluster of amplicons linked to the bead may include a first plurality of copies derived from a first strand of the fragment and a second plurality of copies derived from a second strand of the fragment, complementary to the first. The copies derived from a first and second strands may be present in the cluster in substantially equal portions. The copies derived from the first and second strands may be independently barcoded. Methods may include conducting an amplification reaction with the complex to create a cluster of amplicons copied from the first copy of the fragment and the second copy of the fragment; and sequencing the cluster to generate sequence reads. Methods may include analyzing the sequence reads to confirm the presence of the first barcode and the second barcode to confirm that the copies derived from a first strand and the copies derived from a second strand were both sequenced.

In some embodiments, the tumor sample comprises a formalin-fixed, paraffin embedded (FFPE) tissue slice and method includes liberating the tumor nucleic acid from the FFPE tissue slice. The first and second variants may be identified as a tumor-specific, phased variant biomarker when: the first variant is found in a fragment of the tumor nucleic acid but not found in a first corresponding position in non-tumor reference genomic information; the second variant is found in the fragment but not found in a second corresponding position in the non-tumor reference genomic information; and the first variant is found within less than about two hundred bases from the second variant in the fragment. In some embodiments, the sequence data comprise a plurality of tumor sequence reads, and the method includes generating the non-tumor genomic data by sequencing genetic material from the patient to generate matched normal sequence reads. In certain embodiments, the first variant and the second variant are identified by aligning the tumor sequence reads to the matched normal sequence reads to identify differences.

The sequence data may comprise a plurality of tumor sequence reads, and the first variant and the second variant may be identified by aligning the tumor sequence reads to a published human genome to identify differences. The first and second variants may be identified as the phased variant biomarker when: the first variant is found in the fragment of the tumor nucleic acid but not found in a first corresponding position in the non-tumor genomic data; the second variant is found in the fragment but not found in a second corresponding position in the non-tumor genomic data; and the first variant is found within less than about two hundred bases from the second variant in the fragment. Methods may include, after performing the recited steps and then after the subject is treated to remove the tumor, obtaining a sample from the subject and sequencing sample nucleic acid from the sample to find the phased variant biomarker as evidence of minimal residual disease (MRD). In some embodiments, the sample is a liquid biopsy sample comprising blood or plasma and the sample nucleic acid is circulating tumor DNA (ctDNA) in the blood or plasma.

Other aspects of the invention provide a method of using linked target capture (LTC) for discovering or selecting a phased variant biomarker for a tumor. The LTC discovery method includes: ligating an adaptor that includes a universal priming site to a fragment from the tumor to produce an adaptor-ligated fragment; capturing the adaptor-ligated fragment with a universal primer linked to a probe by hybridizing the probe to a site within the fragment and hybridizing the universal primer to the universal priming site; extending the universal primer with a strand-displacing polymerase to make a copy of the fragment; generating sequence data from the copy; analyzing the sequence data to identify a first variant and a second variant, relative to the non-tumor genomic data, present on the fragment as a phased variant biomarker specific to the tumor. The LTC discovery method may include, prior to the ligating step, obtaining a tumor sample, extracting nucleic acid from the tumor sample, fragmenting the nucleic acid to produce a plurality of fragments that include the fragment. In certain embodiments, the tumor sample comprises a formalin-fixed, paraffin embedded (FFPE) tissue slice and method includes liberating the tumor nucleic acid from the FFPE tissue slice.

In the LTC discovery methods, the first and second variants may be identified as the phased variant biomarker when: the first variant is found in the fragment but not in a first homologous position in non-tumor reference genomic information; the second variant is found in the fragment but not found in a second homologous position in the non-tumor reference genomic information; and the first variant is found within less than about two hundred bases from the second variant in the fragment.

In some embodiments, the sequence data comprise a plurality of tumor sequence reads, and the LTC discovery method further comprises generating the non-tumor genomic data by sequencing genetic material from the patient to generate matched normal sequence reads. In certain embodiments, the first variant and the second variant are identified by aligning the tumor sequence reads to the matched normal sequence reads to identify differences. The sequence data may include a plurality of tumor sequence reads, and the first variant and the second variant may be discovered by aligning the tumor sequence reads to a published human genome to identify differences.

Methods may include, after performing the recited steps and then after the subject is treated to remove the tumor, obtaining a sample from the subject and sequencing sample nucleic acid from the sample to find the phased variant biomarker as evidence of minimal residual disease (MRD). The sample may be a liquid biopsy sample comprising blood or plasma and the sample nucleic acid is circulating tumor DNA (ctDNA) in the blood or plasma.

### BRIEF DESCRIPTION OF DRAWINGS

FIG. 1 diagrams a method of using linked target capture to detect a biomarker e.g., to detect minimal residual disease (MRD).

FIG. 2 shows a linked target capture workflow

FIG. 3 shows probe-dependent primers that may be used in linked target capture.

FIG. 4 shows probe-dependent primers that include sequencing instrument flow cell binding sequences.

FIG. 5 diagrams a method of using proximity sequencing (Pro-Seq) for discovering a biomarker (the path labelled A represents use of two 5′ linked primers; path labelled B represents use of a primer that is linked to an adaptor).

FIG. 6 shows an embodiment of proximity sequencing (Pro-Seq) in which two linked primers are each extended to copy a strand of a fragment.

FIG. 7 shows an embodiment of Pro-Seq in which a primer is extended to copy a strand of a fragment and a second, linked primer is extended to copy a copy of the other strand of the fragment.

FIG. 8 shows an adaptor-based technique for an embodiment of Pro-Seq.

FIG. 9 illustrates seeding a sequencing cluster with a complex generated by a linked target capture technique.

FIG. 10 diagrams a method of using proximity sequencing to detect MRD.

FIG. 11 diagrams steps of a method of using linked target capture for discovering a phased variant biomarker (e.g., for a tumor).

FIG. 12 shows elements of a system useful for performing methods of the invention.

FIG. 13 shows a phase variant specific probe-dependent primer (pvPDP) according to certain embodiments.

FIG. 14 shows steps of a method for targeted capture of nucleic acids using variant-specific LTC.

FIG. 15 summarizes information flow in LTC.

FIG. 16 illustrates information flow in phased variant specific LTC.

FIG. 17 shows an exemplary use of linked ligation adapters of the invention.

FIG. 18 shows steps of an LLA method using linked ligation adaptors to discover or detect a phased variant biomarker.

### DETAILED DESCRIPTION OF THE INVENTION

The invention provides very sensitive and specific detection methods that used phased variants as markers for the presence of a target of interest such as a medical status or condition. Phased variants, as used herein, refer to at least two genetic loci that vary from a reference, i.e., two mutations (compared to the reference) that are found on one strand of one nucleic acid and thus are in phase, in a sense similar to (but not the same as) what is meant by haplotype phasing. A sample may have multiple variants (or mutations) and even when those variants are from within one gene or from genes from the same chromosome, finding the multiple variants in one sample does not mean that the multiple variants were together on one nucleic acid. For diploid organisms, the multiple variants may come from either of two homologous chromosomes. For polyploid organisms (many plants exhibit multiploidy) or aneuploid individuals (e.g., humans with trisomy 21), multiple variants from different homologous chromosomes may be found. As used herein, phased variants refers to at least two variants that are found on one single strand of nucleic acid.

There are circumstances other than polyploid organisms and aneuploid humans in which finding variants together on one strand, i.e., finding phased variants, is specific to a sample or target of interest to a far greater degree than simply finding two variants together in one assay or from one sample. In a few certain sample types or sample conditions, the sample-specificity of phased variants is particularly valuable, given the nature of the sample type or condition, and those situations include highly damaged or degraded DNA, cell-free DNA, complex tumor clonality, and sample preparation workflows that are themselves error-prone, for example, where target amplification by PCR may introduce apparent polymorphisms only as polymerase error.

The sample-specificity of phased variants lies in the fact that any specific mutation will occur only according to some low probability and the chance of finding two such mutations on the same strand is the product of their respective probabilities. So, if PCR is being used to detect polymorphisms, and if the PCR has a 1 in 1,000,000 chance of introducing any given polymorphism, if one is probing for a SNP, after a number of cycles of PCR, the workflow has most probably introduced at least one copy of the SNP via PCR error. However, if one is probing for two SNPs as phased variants, then the PCR is likely to recapitulate, or mimic, those with a probability of 1 in (1,000,000x1,000,000), with the consequence that finding those phased variants in a sample is strong evidence that the sample contained the phased variant biomarker that was being looked for. Thus it may be valuable and informative to established that a set of phased variants is specific to a sample of interest and then use the phased variants as a biomarker to identify nucleic acid specifically from that sample of interest.

For example, in cases of highly damaged or degraded DNA, such as preserved specimens that may include organisms in museum collections or formalin-fixed, paraffin embedded (FFPE) tissue slices, one may be presented with a situation in which the identity of a source sample is known (a particular organism or particular tumor), but the sample yields only a small amount of DNA or DNA that is damaged or fragmented. In such cases, it may be most productive to sequence what DNA is available and analyze the results (e.g., compare the sequence reads to a reference) and specifically find and use phased variants—multiple variants appearing together on one strand from the sample. Methods for identifying or selecting phased variants from such sample are presented herein.

In other examples, one may be working with cell-free DNA (cfDNA) such as fetal DNA in maternal blood or plasma or circulating tumor DNA (ctDNA). In some cases, one may have a tissue sample for a “day 1” assay to discover a sample-specific biomarker, but one may be doing so in order to work from cfDNA samples, such as liquid biopsy samples, at some later date. Because the later liquid biopsy sample may have a relatively small quantity of short target DNA fragments among more abundant non-target cfDNA, knowledge of a target-specific, phased variant biomarker and the linked target capture and/or proximity sequencing technologies herein may provide for a very effective and sensitive test for the target among the non-target material.

Another example in which phased variants may be invaluable as a biomarker involves complex tumor clonality (or, similarly, polyploid plants e.g., within agricultural applications). A sample may ostensibly arise from a diploid organism, but due to tumor clonality, any given locus may exhibit more than two different variants. Stated differently, if two or more variants are different loci are found, those variants in a diploid sample would only have arisen on one of two strands, but in a tumor sample, those variants may be found across multiple homologous strands from multiple tumor clones. In such a situation, phased variants are a very sensitive biomarker for the tumor because, even with a complex tumor clonality, once two variants are found both on the same strand, it is highly improbable to find that pattern of phased variants on material not from the same tumor clone.

Those situations—highly degraded sample, cfDNA, and tumor DNA—and the issues surrounding them raise additional challenges when considering error-prone sample preparation workflows. Some of the most common workflows for analyzing DNA from sample, whether for an initial biomarker discovery assay or when using a biomarker to detect a target in a sample, involve preparing a sequencing library (“library prep”). Common library prep techniques involve one or more polymerase reactions, such as amplification by polymerase chain reaction (PCR), and those reactions are error prone. PCR and other aspects of sample preparation are understood to involve techniques that introduce false variants, sometimes referred to as sample preparation artefacts or polymerase errors. Such artefacts are “variants” in the sense that they do not perfectly match reference genomic information, but they are introduced during sample preparation. Such artefacts are problematic because they may appear to be false positive either during an initial biomarker discover assay or a later test for the presence of the target.

The invention provides detection methods that use phased variants as biomarkers that are specific to a target of interest and the invention also provide methods for detecting or identifying phased variants from a known sample for use as a phased variant biomarker in later sample-detection assays. In view of the potential for sample preparation artefacts to appear as false positives and in view of the small amount of target fragments in certain sample types, methods of the invention use specific techniques such as proximity sequencing and/or linked target capture to discover phased variant biomarkers from known sample and/or to later detect target material in a sample by detecting the presence of the phased variant biomarkers.

Proximity-Sequencing (Pro-Seq) is a method useful for detecting true genetic variants within a sample and distinguish those variants from sample preparation artefacts. While there are different implementations of Pro-Seq, Pro-Seq techniques use one primer linked to another or an adaptor to make linked copies from a nucleic acid fragment (neither being a copy of the other) that have substantially the same sequence, are in the same orientation, and are linked together. Initially, the linked copies may appear as clonal amplicons linked together but they originate in a manner different than by amplification onto a bead. Pro-Seq may use at least two linked primers to make substantially identical first and second copies from the same fragment. Those two copies may come from independent extensions over one strand of a fragment, or one copy may be a copy of a first strand of the fragment and other copy may be a copy of a copy of the second strand. At no time was the first copy copied to make the second copy and at no time was the second copy copied to make the first copy. Pro-Seq was introduced in Pel, 2018, Duplex Proximity Sequencing (Pro-Seq): A method to improve DNA sequencing accuracy without the cost of molecular barcoding redundancy, PLoSOne 13(10):e020266 and its Supporting Information, the contents of which are incorporated by reference. Pro-Seq merges multiple independent copies of each template into a single sequencing read by physically linking the molecular copies and using those independent copies to seed a single sequencing cluster.

In Pro-Seq, multiple independent copies of a template (or fragment) are made and physically linked together. One such copy may be a “sense” strand or “first” strand of a fragment and another such copy may be a copy of an “antisense” or “minus” strand of the fragment. Such embodiments may be performed using a linking adaptor (a y adaptor in which a first strand of the adaptor is linked to a primer that is annealed to a second strand of the adaptor). After attaching and primer extension, the linking adaptor provides a complex in which a first strand of the fragment is linked to a copy of a second strand. Those two strands will have substantially the same sequence in the same 5′ to 3′ orientation (i.e., “in phase”). Other embodiments shown herein use linked primers to make multiple copies in phase. Those multiple in phase copies are independent, meaning that no laboratory (or in vitro) polymerase extension reaction that was used to create one strand or copy was also used to create another. Those multiple in phase copies are intended to be identical (and in phase) and but for any polymerase errors are identical (e.g., are at least substantially identical). Those substantially identical copies may not match each other at positions where the original fragment contained a lesion (e.g., such as an abasic site or pyrimidine dimer) which are lesion types that are meant to be, and that can be, detected using the built-in error correction of Pro-Seq. The complex (multiple substantially identical in phase copies from one fragment) may be treated as a single strand and used to seed a sequencing workflow. E.g., a plurality of copies of a primer linked to a solid substrate (e.g., surface of a flow cell or bead) may anneal to those copies and be extended to form a cluster.

Because multiple DNA copies of the same template are compared for consensus within the same cluster, sequencing accuracy is improved without the requirement of redundant reads. Certain embodiments of Pro-Seq include making a first copy of one strand of a DNA fragment and making linked second copy of a copy of a second strand (complementary to the first) of the DNA fragment, in which the first and second copies are linked at their 5′ ends and have substantially the same sequence. Those linked copies may be amplified to generate a cluster of amplification products for sequencing. By those means, it is possible to represent both senses of the starting duplex in a single sequencing cluster. The cluster is sequenced to generate sequence reads and a consensus of those reads may be analyzed to identify any phased variants present in the starting fragment. Polymerase error or other amplification artefacts will not appear across the entire cluster and thus not as consensus among the resulting reads because neither first copy or the linked second copy were ever copied from the other.

Linked target capture (LTC) is a method of target enrichment that allows one to capture and amplify a specific target sequence from within a sample using one or more primers that are linked to target-specific probes. For LTC, sample nucleic acid may be fragmented and adaptors are ligated to sample fragments. The adaptors include universal priming sites. Any fragmentation and adaptor ligation may be indiscriminate as target-specificity will come in when the primer-dependent probes are introduced. Each primer-dependent probe includes an oligo probe than will anneal to a target of interest and that is linked to a universal primer. By design and has been shown by experimental results, the universal primer will anneal to the universal priming site in the adaptor but only when the probe anneals to its target in the fragment. The probe may be provided as an oligo that is blocked from being extended, and the annealed primer is preferably extended from within the priming site and through the fragment using a strand-displacing polymerase to make a copy of the fragment. LTC is beneficial where one has a locus suspected to harbor a phased variant biomarker because the probe(s) may be designed to anneal within the locus, but the copying (by polymerase) is initiated outside of the locus from the ligated adaptor. The polymerase will copy any variants within the fragment. Due to the universal priming sites annealed at both ends of the fragment, any copy of the fragment will have universal priming sites at both ends and may be amplified using pairs of universal primers. LTC is beneficial because the target specificity of the probes means that only loci of interest will be copied by the probe-dependent primers. Thus where a locus is known to harbor, or suspected to harbor, phased variants (e.g., phased variants identified by a prior phased variant discovery assay or to discover phased variants, e.g., by probing mutational hotspots), LTC provides a target-specific target enrichment technique that generates amplicons with universal priming sites at both ends and is well suited to library preparation for sequencing based assays. LTC is described in Pel, 2018, Rapid and highly-specific generation of targeted DNA sequencing libraries enabled by linking capture probes with universal primers, PLoSOne 13(12):e0208283 and Supplemental Information, the contents of which are incorporated by reference. In summary, LTC provides a targeted sequencing library preparation method, which can replace typical multi-day target capture workflows with a single-day, combined ‘target-capture-PCR’ workflow. As stated, LTC uses target-specific capture probes linked to primers where, because target specificity comes only from the probe, LTC is well-suited for interrogating a sample for phased variants because each capture event and product is specific to one strand and the presence of multiple variants along the one strand has minimal effect on target capture.

Methods of the invention have particular utility in certain disease discovery and analysis applications and particularly in oncology, where phased variants provide an exquisitely tumor-specific biomarker. It has been understood that successful cancer treatment is well served by having knowledge of a tumor-specific biomarker. Some approaches have involved analyzing tumor DNA (DNA known to come specifically from a tumor) for a tumor mutation that is tumor-specific, i.e., found in the tumor DNA but not at a homologous location in non-tumor DNA. The tumor-specificity may be provided by sequencing both tumor DNA and so-called “matched normal” DNA from the same patient and comparing the results to find variants specific to the tumor. The tumor-specificity may come from sequencing tumor DNA and comparing the results to other reference genomic data such as one or more published human genome or to a tumor mutation database or to a cancer genome atlas or similar. Conventional approaches have treated a tumor-specific small mutations such as a single nucleotide polymorphism (SNP) or small (<50 base) insertion or deletion (InDel) as a tumor biomarker with the idea that subsequent detection of the tumor biomarker provides evidence of the tumor in the patient. Unfortunately, sample preparation workflows may be error prone and may introduce sample preparation artefacts such as polymerase errors that are difficult to distinguish from tumor-specific mutations. Problems with sample preparation artefacts may arise at either or both of two distinct points along a cancer discovery, treatment, and analysis journey. As cancer is initially diagnosed or discovered, one may obtain tumor DNA and perform an initial biomarker discovery assay—from the tumor DNA—to discover a tumor biomarker. However, a sample preparation artefact may create a false positive. Once a tumor biomarker is discovered and known, later, subsequent assays may involve interrogating a patient sample for that biomarker as evidence of the presence of the cancer. There, a sample preparation artefact could contribute to a false negative or positive.

An insight of the disclosure is that phased variants offer a level of specificity for which Pro-Seq or LTC have no more than a vanishingly small chance of recapitulating due to the stochastic nature of sample preparation e.g., polymerase error. For example, using Pro-Seq for biomarker discovery means that, given that two copies (neither a copy from the other) seed one sequencing reaction, there is almost no chance that polymerase error will contribute a polymorphism that is mis-interpreted as a true mutation and any phased variants apparent from a Pro-Seq discovery assay are true tumor-specific phased variant biomarkers. Similarly, because the PDPs of LTC reliably capture only the intended target fragments into a sample preparation reaction including genomic material from areas of the fragment outside of a probe-binding site, LTC can be used to discover any phased variant biomarkers and is particularly well-suited to pulling fragments of cfDNA from samples such as liquid biopsy samples. As will be seen, the invention provides specific methods that use: (i) LTC to detect a previously-discovered phased variant biomarker as evidence of the presence of a condition; (ii) Pro-Seq to detect a previously-discovered phased variant biomarker as evidence of the presence of a condition; (iii) Pro-Seq to discover a tumor-specific phased variant biomarker from a tumor sample for later detection; (iv) LTC to discover a tumor-specific phased variant biomarker from a tumor sample for later detection; and (v) combinations of those using LTC and/or Pro-Seq to discover or detect phased variant biomarkers.

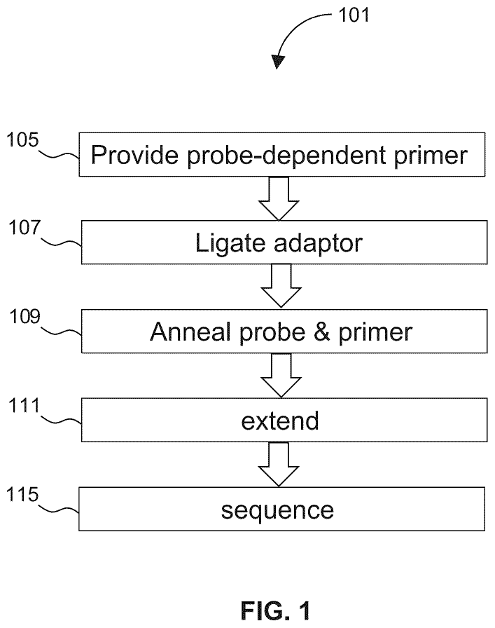

FIG. 1 diagrams a method 101 of using LTC to detect a biomarker e.g., to detect minimal residual disease (MRD). The method 101 includes ligating 107 adaptors to fragments from a sample from a subject. The adaptors include universal priming sites. The method 101 also includes providing 105 a universal primer linked to a probe designed to anneal to a segment of DNA harboring first and second variants that are (i) specific to a tumor of a subject, and (ii) were found present together on one strand of nucleic acid from the tumor. The probe-linked primer may be described as a probe-dependent primer (PDP). The probe of the PDP is hybridized 109 to the fragment and the linked universal primer is hybridized to the universal priming site. The method 101 proceeds by extending 111 the universal primer with a strand-displacing polymerase to make a copy of the fragment; sequencing 115 the copy of the fragment to generate sequence data; and analyzing the sequence data to detect the first and second variants on the fragment to thereby indicate the presence of the tumor in the subject.

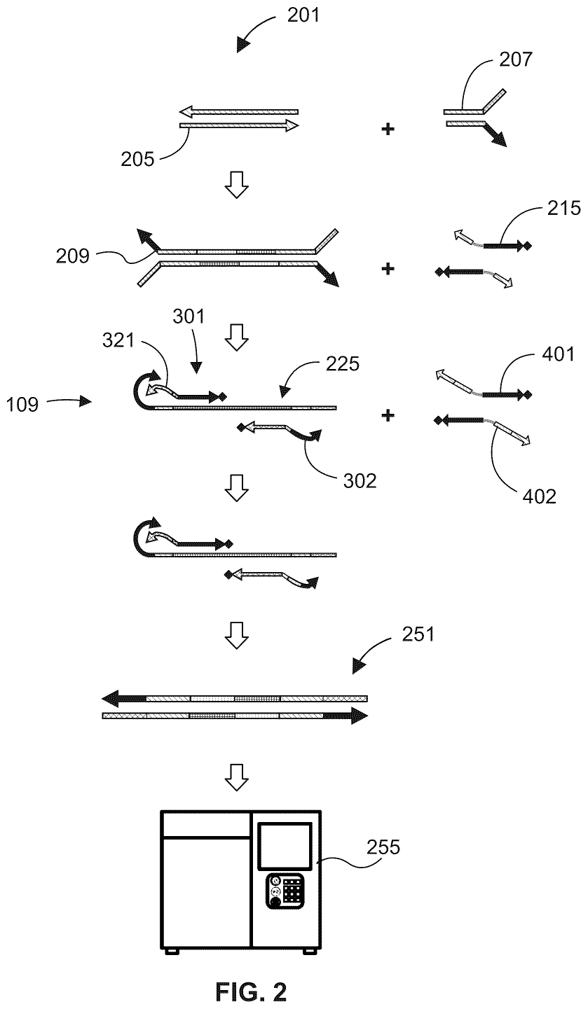

FIG. 2 shows an LTC workflow 201 according to embodiments the method 101. The adaptors 207 will be ligated to the fragment 205. As used herein throughout, arrowheads indicate a 3′ end of a strand. The fragment 205 includes phased variants. In phased variant discovery assays, the presence of the phased variants in the fragment 205 is not known ahead of time, but is discovered by performing the assay. In detection assays, the first and second variants were previously found (e.g., in a discovery assay) as phased variants on one strand of the fragment 205.

The adaptors 207 may be forked, or “Y-adaptors”, and may include a double stranded portion that gets ligated to fragment 205 as well as single stranded portions that may include distinct universal priming sites and also prevent ligation of additional adaptors to the adaptor 207. Once the adaptors 207 are ligated to the fragments to produce adaptor-ligated fragments 209, probe-dependent primers (PDP) 215 are introduced to the reaction mixture. As shown by the arrowheads, each PDP includes two oligos linked at their 5′ ends so that their 3′ ends point away from each other.

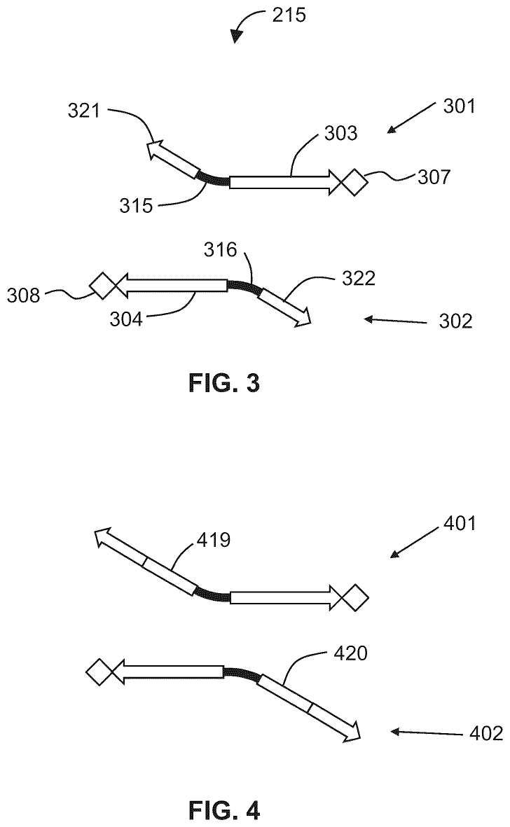

FIG. 3 shows the PDPs 215. Shown as a pair, the PDPs 215 include a first (or “forward”) PDP 301 and a second (or “reverse”) PDP 302. Each PDP includes two oligonucleotide segments. The first PDP 301 includes a first target specific probe 303 that is provided by an oligonucleotide designed to anneal to the target fragment 205. The first PDP 301 includes a first universal primer 321 that is provided by an oligonucleotide designed to anneal to a universal priming site in the adaptor 207. A 5′ end of the first universal primer 321 is linked to a 5′ end of the first target specific probe 303 by a first linker 315 which may be provided by any suitable material such as poly-ethylene glycol (PEG), a bis-maleimide PEG linker or click chemistry, a solid substrate such as a bead or particle or a surface of, e.g., a slide, a flow cell, or a well of a well plate. A 3′ end of the first target specific probe 303 includes a 3′ blocker 307, which is any group or modification that prevents extension of the probe 303 by a polymerase in a template dependent fashion. Suitable blockers 307 may include an inverted base, a modified base, a 3′ de-oxy carbon, a phosphorothiate linkage, or any other group or modification.

The PDPs 215 may include a second (e.g., “reverse”) PDP 302 that includes a second target specific probe 304 (e.g., an oligonucleotide designed to anneal to the target fragment 205 on a strand complementary to where first probe 303 anneals); a second blocker 308; and a second universal primer 322 designed to anneal to a universal priming site in the adaptor 207. A 5′ end of the second universal primer 322 is linked to a 5′ end of the second target specific probe 304 by a second linker 316. It is noted that the second PDP 302 is optional and that linked target capture may be performed using just a first PDP 301.

Referring back to the LTC workflow 201, the first PDP 301 anneals to the adaptor ligated fragment 209 (as does the second PDP 302). Specifically, the first target specific probe 303 anneals to the target fragment 205 as designed and the first universal primer 321 anneals the universal priming site in the adaptor 207 of the adaptor ligated fragment 209. The first universal primer 321 is extended by a polymerase to copy the adaptor ligated fragment 209, including the target site where the probe anneals and the universal priming sites at both ends. Similarly, the second universal primer of the second probe 301 is also extended to copy the other strand of the adaptor ligated fragment 209.

Extension of the probes of the PDPs 215 creates a plurality of copies 225 of the original fragment (to which the probes are complementary) with universal priming sites at both ends. Certain preferred embodiments use a second pair of PDPs 401, 402 that include sequencing instrument flow cell binding sequences.

FIG. 4 shows the pair of PDPs 401, 402 that include sequencing instrument flow cell binding sequences 419, 420. The second PDPs 401, 402 are similar to the PDPs 215 except that the second PDPs 401, 402 may include flow cell binding sequences 419, 420.

Referring back to the LTC workflow 201, it is noted that the steps may be performed using both of PDPs 215 and second PDPs 401, 402, or may be performed using only one pair of the PDPs (e.g., PDPs 215 or second PDPs 401, 402) or may be performed using only a single PDP, e.g., PDP 301 (to initiate a linear amplification). Even if the steps are performed with only a single pair of PDPs 215 or only a single PDP, due to the inclusion of the universal priming sites, the product may be amplified with primers that add flow cell binding sequencing, to provide a sequencing library 251. The sequencing library 251 includes a plurality of copies of a target (selectively enriched for by at least the first probe 303) with sequencing instrument binding sequences (such as Illumina adaptor sequences) at both ends.

As noted, in the method 101, the first probe 303 of the PDP 301 is hybridized 109 to the fragment and the first universal primer 321 is hybridized to the universal priming site after which the method 101 proceeds by extending 111 the universal primer with a strand-displacing polymerase to make a copy of the fragment. It has been found that the primer extension reaction only occurs when the probe anneals to its intended complementary target in the adaptor-ligated fragment 209. It may be theorized that, when the probe 303 anneals to the fragment 209, the primer 321 behaves as if it were present in much higher concentration, because the linker 315 keeps the primer 321 in proximity to the universal priming site. Results with the depicted molecular reagents have shown that the primer 321 is only extended when the probe 303 is annealed to its target in an adaptor ligated fragment 209. Because results and experience with linked target capture techniques have shown that extension of primer 321 to copy the adaptor-ligated fragment 209 occurs only when the probe 303 is annealed or hybridized to the target in the adaptor-ligated fragment, the extension 111 step in the method 101 may be described as extending 111 the universal primer of the PDP when the probe of the PDP is annealed to its target in the adaptor-ligated fragment. Evidence and results have shown that the universal primer does not extend from universal priming sites on adaptor ligated fragments for fragments that do not include the target site cognate to the (blocked) probe.

Methods of the disclosure make use of universal primers and universal priming sites. As used herein, a “universal primer” refers to an oligo that functions as a PCR primer and having a sequence complementary to a designed, or human-made, universal priming site. Within any workflow, there may be more than one distinct universal primer employed. The LTC workflow 201 that is shown includes the use of four distinct universal primers (respectively provided by first forward PDP 301, first reverse PDP 302, second forward PDP 401, and second reverse PDP 402). In any such case, each universal primer has a sequence unique from the others and complementary to the universal priming site to which that universal primer anneals. The universal priming sites are designed, or human made, just like the universal primers. In substantially all cases, a universal primer and a universal priming site will not anneal to a locus in naturally-occurring genetic material (i.e., by design). A person of skill in the art is familiar with the use of universal primers and their priming sites, the design of those elements to only anneal (or hybridize) to their intended cognates and not to genomic DNA. To illustrate, making reference to the hybridizing 109 step shown in the LTC workflow 201, the skilled artisan understands that the first universal primer 321 anneals, by design, to a cognate priming site in one strand from the adaptors 207 (and nowhere else) while the second universal primer 322 of the second PDP 302 will only anneal to the other universal priming site in the other stand from the adaptors 207.

Due to the target specificity of any of the probes 303, 304 etc., the method 101 will only amplify adaptor ligated fragments 209 that contain a target sequence of interest, e.g., a genomic region that is suspected to harbor a specific variant such as a set of phased variants. The LTC workflow 201 may be used to generate a sequencing library 251 that includes a plurality of copies of the target that optionally include binding sequences for a sequencing instrument 255. The method preferably includes sequencing the target to determine the presence of phased variants.

The method 101 uses LTC to detect phased variants in a sample. That is, the method 101 is useful where the phased variants have previously been discovered as a biomarker for a sample of known properties (e.g., a tumor sample). Once the phased variant biomarker is discovered as a tumor or other sample-specific biomarker (optionally using other methods of this disclosure), the method 101 is used to detect the phased variant biomarker as evidence of a condition such as minimal residual disease (MRD).

For example, in some embodiments, a subject has been diagnosed with cancer and LTC and/or Pro-Seq are performed on a sample from a tumor from the subject (e.g., a tumor biopsy or FFPE tissue slice). The LTC and/or Pro-Seq are used to discover a set of phased variants that is specific to the tumor (i.e., not found in the genetic material of non-proliferative cells, i.e., healthy cells, of the subject). The phased variant information is stored for later use. The subject is treated to remove the tumor. Later, a sample from the subject may be interrogated using the method 101. The target probes are designed to capture a DNA fragment that would harbor the phased variants if present. Certain preferred embodiments use a liquid biopsy sample, e.g., a blood draw, after treatment (e.g., days, months, and/or years after treatment). With liquid biopsy, a blood draw may be performed and the blood sample may be treated by known methods to extract or separate any cell-free DNA (e.g., centrifuged to separate whole cells followed by DNA purification using a commercially available kit). It is understood that cfDNA from a blood or plasma sample has a modal fragment size of about 170 bases. That cfDNA is subject to adaptor ligation and the LTC workflow 201. Sequencing on instrument 255 generates sequence data. In MRD embodiments, because the phased variants are known and are extremely unlikely to arise together by chance as a sample preparation artefact, the sequence data may simply be analyzed (e.g., scanned by a computer program) for the presence of phased variants, where positive detection of phased variant biomarker in the sequence data is evidence of MRD in the subject.

For those purposes, phased variants are considered to be any at least two variants, relative to reference genomic information, that are found together on one DNA strand less than certain distance (e.g., about 200 bases, optionally less than about 170 bases, or 100 bases, or 80 bases, or some other such number of bases) apart. By finding two or more variants, specific to tumor DNA, less than about e.g., 200 or 170 bases apart, in tumor DNA, then finding those phased variants together on one strand of cfDNA from a liquid biopsy sample is evidence of the presence of MRD in the subject.

As noted, the method 101 uses the phased variant biomarker, once known, to detect evidence of a condition (e.g., MRD). Methods may include, prior to the detection, performing a phased variant discovery process of using linked target capture (LTC) for selecting the phased variant biomarker for the tumor. Using LTC for phased variant discovery may include ligating an adaptor comprises a universal priming site to tumor nucleic acid from a tumor sample from the subject to produce an adaptor-ligated fragment; capturing the adaptor-ligated fragment with a universal primer linked to a probe by hybridizing the probe to a site within the tumor nucleic acid and hybridizing the universal primer to the universal priming site; extending the universal primer with a strand-displacing polymerase to make copies of the fragment; and sequencing the copies. Those steps are essentially similar to the LTC workflow 201 except that: (i) the starting sample is known to be a tumor sample, and (ii) analysis of the sequence data includes comparing the initial sequence data to known, non-tumor reference genomic data to discover phased variants specific to the tumor.

To (i) start with a tumor sample may include obtaining a sample such as a tumor biopsy or FFPE tumor slice. With such a sample, DNA is extracted or liberated (e.g., from the paraffin) and copied using LTC to produce a tumor-specific sequencing library.

To (ii) analyze sequence data to discover phased variants specific to a tumor involves sequencing the tumor-specific sequencing library. Sequencing will generally produce a large number of sequence reads. For example, operating an instrument that performs short-read sequencing by synthesis such as the next-generation sequencing (NGS) instrument sold under the trademark HISEQ or MISEQ by Illumina will generate a large number of short reads, typically output in a FASTA or FASTQ file format. Similar results may be obtained using a sequencing instrument from Roche, IonTorrent, or Ultima Genomics. Those sequence reads may be trimmed and/or cleaned up prior to informatics analysis. Those sequence reads may be assembled, e.g., by de novo assembly, mapping to a reference, or some combination thereof. The assembled sequence reads may be compared to (“mapped to”) a reference to find variants (positions where a sequence read is homologous to, but does not match, the reference). Because an LTC phased variant discovery assay starts with making adaptor-ligated fragments, there may be no need to set a threshold distance apart for defining phased variants. Instead, any set of sample-specific variants that arise from the same amplified fragment are known to have been present together on the same contiguous DNA strand and may be taken as a phased variant biomarker for that sample.

As noted, the method 101 uses the phased variant biomarker, once known, to detect evidence of a condition (e.g., MRD). Methods may include, prior to the detection, performing a phased variant discovery process of using proximity sequencing (Pro-Seq) for selecting the phased variant biomarker for the tumor.

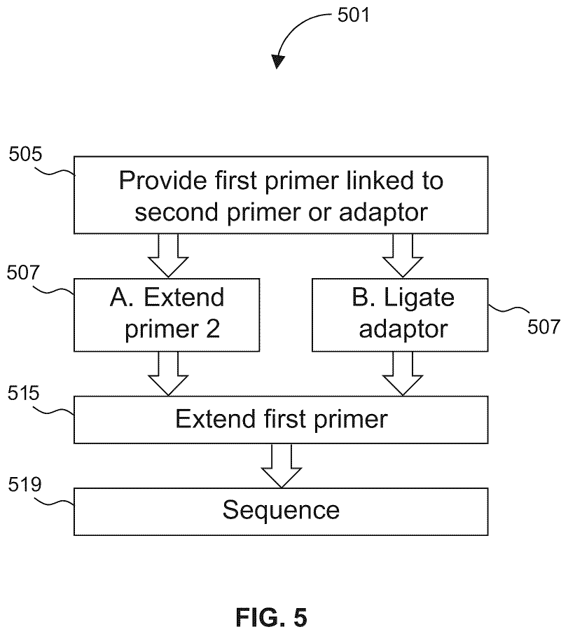

FIG. 5 diagrams a method 501 of using proximity sequencing for discovering and/or selecting a biomarker specific for a condition such as a phased variant biomarker for a condition such as cancer. The method 501 includes exposing a nucleic acid fragment, or copies thereof, to (i) a primer that is linked to a second primer (embodiment A), or to (ii) a primer that is linked to an adaptor that will be ligated to the fragment (embodiment B). In embodiment A, two linked primers will each be extended 507 to make copies of a fragment. Those copies will be linked to each other (because the primers are linked to each other) and those two copies will have substantially the same sequence and same orientation. Those two copies can have substantially the same sequence under two possible workflows. In the first workflow of embodiment A, each one of the linked primers makes its own copy of a strand of the fragment. That is, the first primer is annealed to the strand and extended, and then the second primer is annealed to the strand (after the first primer and its copy are melted away) and extended. In the second workflow of embodiment A, the first primer (of the linked primers) anneals to one strand of the fragment and is extended to make a copy of the one strand. A reverse primer is annealed to a second strand (the reverse complement of the first) and is extended to make a copy of the second strand after which the second primer (linked to the first) is annealed to the coy of the second strand and extended to make a copy of the copy of the second strand. In embodiment B, the fragment is ligated to an adaptor and the adaptor includes on strand that is ligated to one strand of the fragment and is linked to a primer that anneals to the other strand of the fragment. In this embodiment B, the method 501 includes ligating 508 the adaptor to at least one strand of the fragment and annealing the linked first (e.g., only) primer to the other strand. In all embodiments, the method 501 includes extending 515 the first (or only) primer.

The product of the extension and optional adaptor ligation is a complex that includes a strand of the fragment or copy thereof linked to a primer extension product that includes a copy of the strand or a copy of a copy of the other strand. In each embodiment and workflow, the two linked nucleic acids will have substantially the same sequence (essentially identical allowing for polymerase error or other imperfections). Notably, neither linked strand of the complex is a copy (direct or indirect) of the other. Each linked strand is from the fragment or made by copying from the fragment and any polymerase reaction in the production of one strand of the complex does not contribute to production of the other strand. As a consequence, any polymerase error that changes a base in an extension product (relative to the original fragment) will not appear in both strands of the complex.

The preceding is written to introduce the genesis of a complex that includes two substantially identical strands for use in Pro-Seq, but the complex may include any number of strands. In some embodiments, a first strand and a copy of a second strand of a fragment are amplified onto beads that are decorated with primer (e.g., hundreds or thousands of primers) to generate a complex of bead-bound amplicons all having substantially the same sequence and all linked to the beads in the same informatic orientation (e.g., so that the sequences of the linked strands all substantially align to one another).

As mentioned, Pro-Seq may use one of several workflows including: a first version of embodiment A (two linked primers each extended to copy a strand of a fragment); a second version of embodiment A (a primer extended to copy a strand of a fragment and a second, linked primer extended to copy a copy of the other strand); and embodiment B (strand of a fragment is ligated to adaptor that is linked to primer that is extended to copy other strand of the fragment).

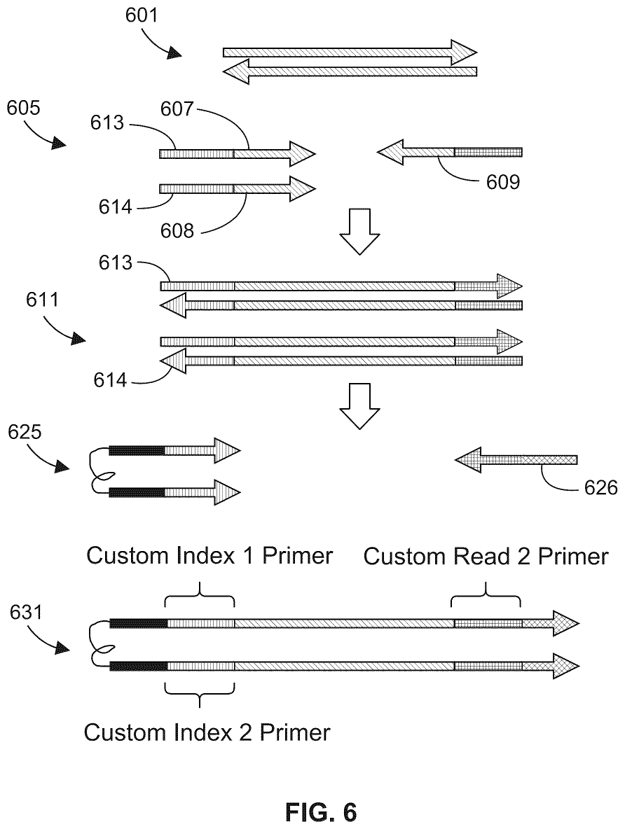

FIG. 6 shows steps of the first version of embodiment A of Pro-Seq in which two linked primers are each extended to copy a strand of a fragment. A fragment 601 is introduced to a set of locus-specific primers 605. The locus-specific primers 605 includes a first forward primer 607, a second forward primer 608, and a reverse primer 609. The first forward primer 607 and the second forward primer 608 both have an identical 3′ locus-specific targeting sequence that will anneal to the fragment 601 and they have different universal priming sites in their 5′ tails, e.g., first universal priming site 613 and second universal priming site 614. The reverse primer 609 also has a locus specific 3′ segment and a 5′ tail with a universal priming site.

Those elements are subject to an amplification reaction to generate two similar, but distinct, sets of amplicons 611. The amplicons 611 include copies from both strands of the fragment 601 and are substantially identical except that some of the amplicons 611 include the first universal priming site 613 and some include the second universal priming site 614. The copies are identical in the sense that they would be identical but for any artefact introduced by the sample preparation. If the copying or amplification reaction introduces a base change, or polymerase error, then the amplicons will still be nearly identical, aka substantially identical. An important feature is that such an artefact will only show up in some, not all of the amplicons 611.

The amplicons 611 are amplified with a set of linked, tailed universal primers 625 and a tailed reverse primer 626 to produce a complex 631 that includes first and second copies of the fragment 601. Notably, due to the linked, tailed universal primers 625 having different first universal priming site 613 and second universal priming site 614, those primers only annealed to copies from distinct first-strand synthesis reaction with the locus-specific primers 605. Due to that, the two strands in the complex 631 were each made without copying a copy that was made in the production of the other strand of the complex. Due to statistical chance, it is observed that on average, about 50% of the two primer complexes will include copies from each sense, or complementary strand, of the starting duplex.

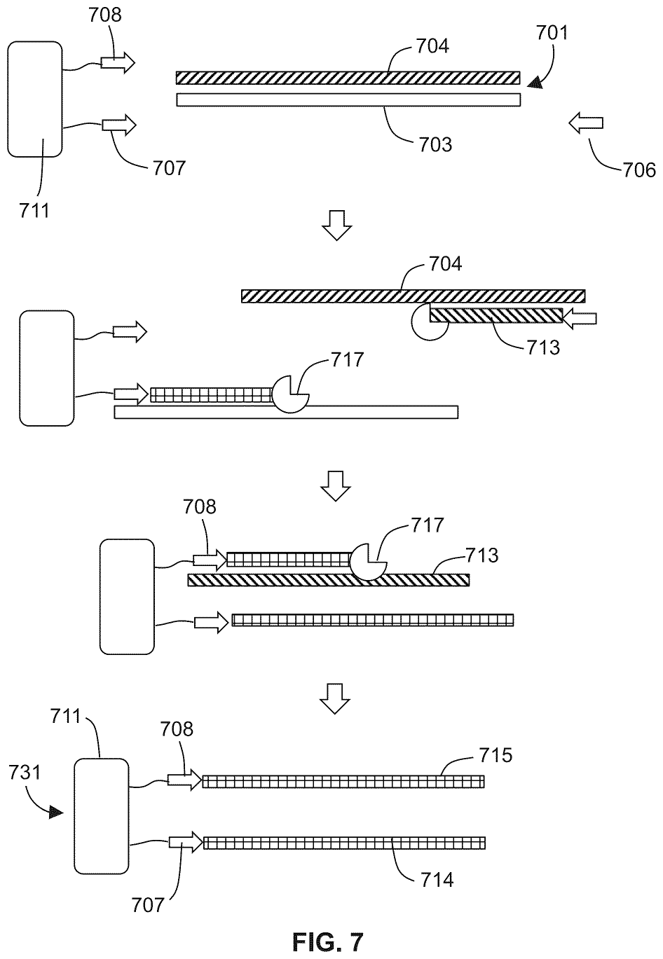

FIG. 7 shows steps of the second version of embodiment A of Pro-Seq in which a primer 707 is extended to copy a strand 703 of a fragment 701 and a second, linked primer 708 is extended to copy a copy 713 of the other strand 704 of the fragment 701. After primer 708 is extended by polymerase 717, the result is a complex 731 that includes: a linker 711, the first primer 707, the second, linked primer 708, a copy 714 of the strand 703 of the fragment 701 and a copy of a copy 715 of the other strand 704 of the fragment. Neither copy in the complex 731 was made by copying any intermediate in producing the other copy of copies 714, 715. Thus, if polymerase error or other reaction noise introduced an artefact when copying from fragment 701, then the artefact will only appear in one of copy 714 and second copy 715.

There are several mechanisms by which to ensure that the complex 731 includes copies originating from both strands of the fragment 701. In one embodiment, a reverse primer 706 is introduced and a limited number of thermocycles are conducted to promote linear (non PCR) copying of the other strand 704 to generate copy 713, after which the fragment 701 and copies 713 are subject to amplification with an abundant number of first and second linked primers 707, 708. In another embodiment, the linker 711 is a bead decorated with many (e.g., hundreds to thousands of copies) of the first and second primer 707, 708. Results have indicated that the reaction products are essentially stoichiometric to the inputs and the bead will carry a substantially equal number of the copy 714 and second copy 715. In another embodiment, the fragment 701 is ligated with Y-adaptors (or “forked adaptors”) having first and second universal priming sites in the single-stranded ends. The first and second primers may be, respectively, specific for the first and second universal priming sites.

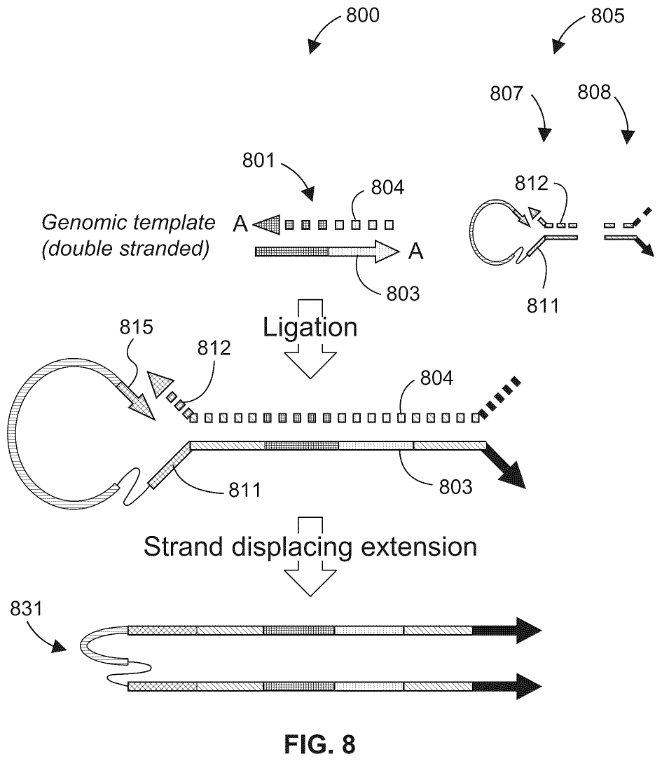

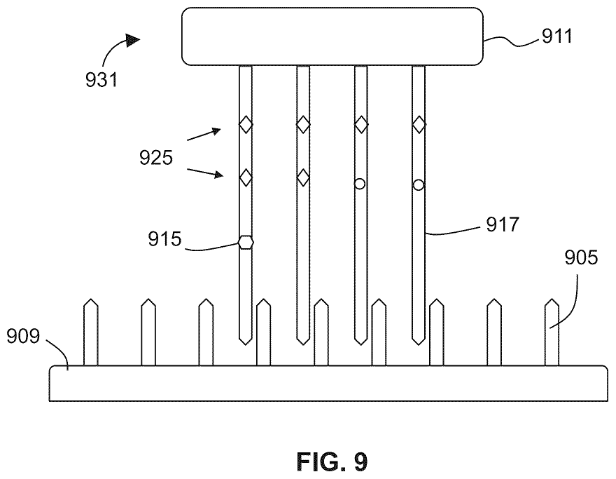

FIG. 8 shows an adaptor-based technique 800 for embodiment B of proximity sequencing (Pro-Seq). A fragment 801 of template DNA is introduced to adaptors 805, which include the depicted first partially double-stranded adaptor 807 and second partially double stranded adaptor 808. The fragment 801 includes a strand 803 and a second, other strand 804 that is complementary to the strand 803. In the depicted steps of embodiment B of Pro-Seq, the fragment 801 (which includes phased variants) is ligated to adaptors 807, 808. The adaptor 807 includes a primer-linked strand 811 that is linked to a primer 815 that is extended to copy the other strand 804 of the fragment. The primer-linked strand 811 (one strand of the adaptor 807) is linked to the primer 815. In a ligation reaction, the 3′ end of the primer-linked strand 811 gets covalently ligated to the 5′ of the strand 803 of the fragment 801, and the 3′ end of the second strand 804 also gets ligated to the 5′ end of the second strand 811 of the adaptor 807 (the second partially double stranded adaptor 808 gets ligated to the other end of the fragment 801). The primer 815 anneals at the 3′ portion of the complementary strand 812 of the adaptor. An extension reaction with a strand-displacing polymerase forms a complex 831. The complex 831 includes the strand 803 of the fragment 801 and a copy of the other strand 804 of the fragment.

Whichever embodiment is used, the method includes producing a complex comprising a first strand, or copy thereof, of the fragment and a second copy of the fragment linked together at their 5′ ends in which the first copy of the fragment and the second copy of the fragment have substantially the same sequence.

Methods of the invention include generating sequence data from the complex.

FIG. 9 illustrates seeding a sequencing cluster with a complex 931 generated by an LTC technique. The complex 931 includes strands 917 that were copied from fragment and the strands are linked (at their 5′ ends) via a linker 911, such as PEG or a bead. The complex 931 may be captured to a solid support 909 by hybrid capture using a plurality of capture oligos 905 immobilized on the solid support. Any suitable solid support 909 may be used such as, for example, a surface of a flow cell or a bead. In some embodiments, the solid support also has second, reverse oligos immobilized thereto (not shown) and the methods include amplifying the strands 917 by bridge amplification prior to conducting a sequencing reaction.

In one set of “bead-based” embodiments, the solid support 909 is a bead, and the method includes copying the copies 917 onto the bead. In other “bead-based” embodiments, the linker 911 is a bead (and there is no separate solid support 909). In the bead based embodiments, amplification generates a cluster attached to the bead. The cluster includes a plurality of copies generated from one fragment in which at least a first portion of the copies and a second portion of the copies were each made by independent sets of polymerase reactions such that a polymerase error or other artefact in one portion of the copies does not appear in the other portion. In preferred embodiments, one portion of the copies 917 is made from one (e.g., “sense”) strand of a fragment, and an other portion of the copies 917 is made from the other (e.g., “antisense”) strand of the fragment.

As shown, the copies 917 may include a true phased variant 925 (at least two mutations, relative to a reference, that appeared in one fragment from the sample) and at least one sample preparation artefact 915. Due to the Pro-Seq workflow, the true phased variant 925 will appear in all of the copies and the artefact 915 will appear in fewer than all of the copies 917.

The cluster of copies 917 may be subject to a sequencing reaction. Any suitable sequencing reaction may be performed. For example, the substrate 909 may be a surface of a flow cell that is loaded into an NGS sequencing instrument from Illumina. In other embodiments, the cluster is attached to a bead, which is interrogated in a well by a pyrosequencing technique. In other embodiments, the bead-bound cluster is subject to a sequencing-by-synthesis reaction in a well in a semiconductor device with a pH meter that detects a pH change as known bases are incorporated and sequence data is written to file by a sequencing instrument such as the semiconductor sequencing platform sold under the trademark IONTORRENT. In other embodiments, the cluster is bound to a bead which is held in a location on a flow cell a sequenced by the incorporation of fluorescently labeled nucleotides with fluorescence read and interpreted by a sequencing instrument such as the one sold under the trademark ULTIMA GENOMICS. The output of the sequencing reaction is a set of sequence data, sometimes stored as a FASTA or FASTQ file. Such sequence data may be assembled and/or aligned to a reference to identify a true phased variant 925 present in the fragment from the sample.

In preferred embodiments of the Pro-Seq methods, the cluster may include copies from one (e.g., “sense”) strand of a fragment, and copies made from the other (e.g., “antisense”) strand of the fragment. The fragments may be barcoded (e.g., via adaptor ligation) to barcode each strand. Using, for example, forked adaptors (or “Y-adaptors”), different barcodes may be in the single stranded portions of the adaptors, providing different barcodes to copies of (or copies of copies of) the first and complementary second strands. Methods may include analyzing the sequence data or sequence reads to confirm the presence of the first barcode and the second barcode to confirm that the copies derived from a first strand and the copies derived from a second strand were both sequenced

As mentioned, the method 501 uses proximity sequencing (Pro-Seq) for discovering and/or selecting a biomarker, such as a phased variant biomarker, specific for a condition such as cancer. This can be done by starting with a tumor-specific sample, such as a formalin-fixed, paraffin embedded (FFPE) tissue slice and method includes liberating the tumor nucleic acid from the FFPE tissue slice. The first and second variants may be identified as a tumor-specific, phased variant biomarker 925 when the first variant is found in a fragment of the tumor nucleic acid but not found in a first corresponding position in non-tumor reference genomic information; the second variant is found in the fragment but not found in a second corresponding position in the non-tumor reference genomic information; and the first variant is found within less than about two hundred bases from the second variant in the fragment. Such identification involves comparing the sequence data from the tumor sample to non-tumor reference genomic information. Any suitable non-tumor reference genomic information may be used.

The non-tumor reference genomic information may be provided by sequencing genetic material from the patient to generate matched normal sequence reads. Comparison of tumor reads to “matched normal” reads is known in the art. Finding the phased variants 925 may involve aligning the tumor sequence reads to the matched normal sequence reads to discover differences. The alignment may be performed automatically, by a computer system. For example, a computer system may implement an alignment algorithm (such as the Burroughs Wheeler alignment) provided by software such as bwa-mem.

The output of the method 501 is a discovered set of phased variants 925 that are specific to a sample, such as a tumor sample. There is special significance to the interplay between the Pro-Seq workflow and phased variants when it is intended to assay cfDNA for the phased variants. A tumor-specific phased variant biomarker may be present in cfDNA in remarkably small quantities, i.e., after tumor resection, minimal residual disease may manifest as a very rare and hard to find mutation in cfDNA. The allele fraction of any variant among cfDNA may be much lower than the frequency of sample preparation artefacts 915 such as polymerase error. Thus, any one polymorphism may be statistically indistinguishable from likely artefacts 915. Also the likelihood of artefacts mean that conventional PCR-based discovery assays are likely to include false positive phased variants (e.g., two polymerase errors introduced during PCR of any starting fragment). However, the chance of sampling artefacts recapitulating a true phased variant when using Pro-Seq are vanishingly small, so a Pro-Seq based discovery assay will find phased variants as biomarkers that can reliably be used later for surveillance for MRD.

Thus, methods may include, after performing the discovery methods 501 and then after the subject is treated to remove the tumor, obtaining a sample from the subject and sequencing sample nucleic acid from the sample to find the phased variant biomarker 925 as evidence of minimal residual disease (MRD).

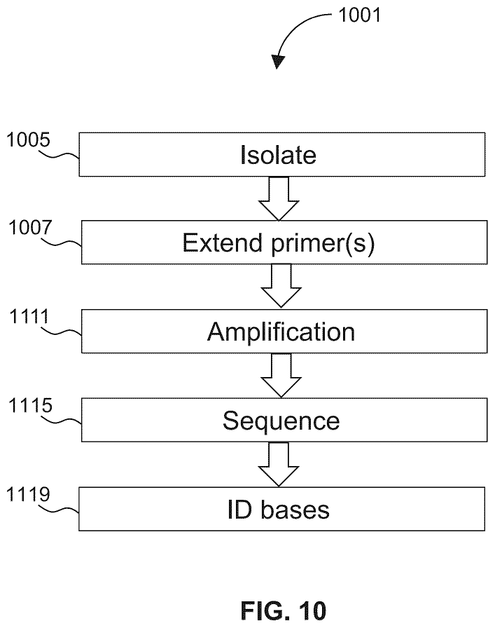

FIG. 10 diagrams a method 1001 of using proximity sequencing to detect MRD. The method 1001 includes isolating 1005 a nucleic acid fragment from a sample from a subject in a reaction volume with a first primer and a second primer that is linked to the first primer. The reaction volume is preferably an aqueous partition such as a droplet (e.g., in an emulsion or electrowetting device), well (e.g., in a multiwell plate or picotiter plate), fluidic harbor, tube, slug (sometimes called a “plug”), or other aqueous or gel volume. The first and second primers are extended 1007. In an alternative, an adaptor that is linked to a primer is ligated to one strand of the fragment, and the primer that is linked to the adaptor copies the other stand of the fragment, as shown in the adaptor-based technique 800 of Pro-Seq, in which a primer that is linked to an adaptor that will be ligated to the fragment (similar to embodiment B of the method 501). Important to the method 1001 is that a phased biomarker is known. The method 1001 is for detecting a phased variant biomarker (that is known) in a sample. The method may be used, for example, to detect evidence of a tumor (e.g., in a test for remission, relapse, metastasis, or minimal residual disease). The method 1001 is well suited to liquid biopsy, where blood is drawn, spun down, subject to DNA extraction for cfDNA, which is then tested by this method 1001. Extending 1007 the first primer and the second primer (or the primer linked to the fragmented-ligated adaptor) produces a complex comprising first and second copies of the fragment (or a strand of the fragment and a copy of the other strand of the fragment) linked together at their 5′ ends. The first copy of the fragment and the second copy of the fragment have substantially the same sequence. If there is no polymerase error or other sample preparation artefact or DNA lesion, the strands of the complex will have the same sequence. However, a point of the method 1001 is the acknowledgement that the recited steps themselves may introduce errors, artefacts, or lesions, with the consequence that strands in the complex may have different, but substantially the same, sequences. The method 1001 includes conducting 1111 an amplification reaction with the complex to create a cluster of amplicons copied from the first copy of the fragment and the second copy of the fragment and sequencing 1115 the cluster to generate sequence reads. A base identity is assigned 1119 to each position in the fragment for which the sequence reads are in consensus. The base calling provides a base sequence of the fragment and that base sequence is analyzed to detect first and second variants that were found to be specific to a tumor of the subject when found present together on one strand of nucleic acid from the tumor. A significant feature of the method 1001 is that the complex include multiple strands that: have substantially the same sequence; are in the same orientation; are used to seed a sequencing cluster or reaction; originate from one fragment; are not copied from each other or from intermediate polymerase copies that lead to the generation of the other. In certain preferred embodiments, the cluster includes one or more strands that originate from one strand of a fragment and one or more strands that originate from the other strand of the fragment.

For example, in some embodiments, a first copy of the fragment is synthesized by a polymerase copying a first strand of the fragment and a second copy of the fragment is synthesized by a polymerase copying a copy of a second strand of the fragment, complementary to the first strand. In certain embodiments, a first primer is linked to a second primer by both being linked to a bead. The bead may have a plurality of primers that are extended to create a cluster of amplicons all linked to the bead (e.g., the cluster of amplicons linked to the bead includes a first plurality of copies derived from a first strand of the fragment and a second plurality of copies derived from a second strand of the fragment). The separate strands of the fragment may be barcoded to give a different barcode to each strand (e.g., by ligating a Y-adaptor to the fragment, or by priming each strand with a different barcoded primer) and analyzing the sequence reads may include confirming the presence of a first barcode and a second barcode to confirm that the copies derived from a first strand and the copies derived from a second strand were both sequenced.

As noted, the method 1001 includes isolating 1005 the fragment in a in a reaction volume. Isolation of a DNA fragment may be performed by extracting DNA from a sample into an aqueous solution. The solution may be diluted and partitioned into aqueous compartments. A so-called limiting dilution or terminal dilution involves diluting the sample to a degree that, once the sample is divided into compartments, a majority of the compartments will have zero or one DNA fragment therein. A small number of compartments may have more than one DNA fragment and, interestingly, those compartments do not really interfere with an outcome of any of the methods here (e.g., phased variants on one fragment are still detected when thousands of aqueous partitions are made and one or a few of those partitions have two or more DNA fragments). Calculating an amount by which to dilute may be performed by measuring a quantity of DNA in an aqueous solution (e.g., by optical density). If mean or modal fragment length is known, then quantity (from OD) allows one to calculate number of molecules, from which the skilled artisan can calculate a dilution factor. Perhaps more significantly, there are known instruments and platforms that partition samples into aqueous compartments (e.g., droplets or wells) at the appropriate dilution.

Noting that the method 1001 is for detecting a phased variant in a sample, e.g., to detect MRD by performing Pro-Seq with cfDNA for a liquid biopsy sample, the method 1001 may optionally also have included, prior to the recited steps, performing an assay to discover the phased variant biomarker in a sample from a known a source or tissue type.

As discussed above, the method 1001 may include (prior to the recited steps), the method 501 that uses Pro-Seq to discover and/or selecting a phased variant biomarker specific for a condition or tissue or sample type.

It is noted that method 1001 uses Pro-Seq to detect a phased variant biomarker and that method 101 uses linked-target capture (LTC) to detect a phased variant biomarker. It is further noted that LTC is well suited for the discovery of phased variants in a sample of known type, origin, or source, such as a tumor sample.

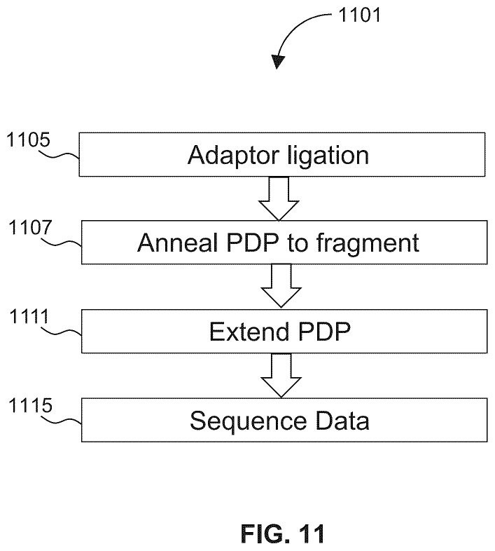

FIG. 11 diagrams steps of a method 1101 of using linked target capture for discovering a phased variant biomarker (e.g., for a tumor). The method includes ligating 1105 an adaptor comprises a universal priming site to a fragment (e.g., from the tumor) to produce an adaptor-ligated fragment. Notably, the adaptor ligation 1105 step may be indiscriminate. All nucleic acid may be extracted from a sample, optionally fragmented (e.g., by shearing, sonication, enzymatically, etc.), optionally blunt-ended, and reacted with excess adaptors and ligase. A benefit of LTC is that LTC is useful to enrich a specific target in a sequence-specific manner. The target specificity does not come at the adaptor ligation 1105 steps, but through the introduction, annealing, and extension of a probe-dependent primer (PDP). Thus, adaptor ligation may be performed “in bulk”, meaning that any and/or all nucleic acid from a sample may be exposed to excess adaptors and ligase to generate one or more adaptor-ligated fragment(s).

The method 1105 then includes capturing 1107 the adaptor-ligated fragment with a universal primer linked to a probe (a probe-dependent primer, or “PDP”) by hybridizing the probe to a site within the fragment and hybridizing the universal primer to the universal priming site. The capturing 1107 step may follow the LTC workflow 201 as show previously. After adaptors 207 are ligated to a fragment 205, probe-dependent primers (PDP) 215 are introduced to the reaction mixture. In certain embodiments, the PDPs 215 include a first (or “forward”) PDP 301 and a second (or “reverse”) PDP 302 as shown previously. In some embodiments, only one PDP 301 is used.

In this method 1105 of using LTC to discover phased variants, at least one fragment 205 includes phased variants. This is not known at the outset of the method 1101, but will be discovered. The method 1101 proceeds by extending 1111 the universal primer with a strand-displacing polymerase to make a copy of the fragment. Sequence data is generated 1115 from the copy and analyzed to identify a first variant and a second variant, relative to non-tumor genomic data, present on the fragment as a phased variant biomarker.

For purposes herein, phased variants includes any two or more variants that are present in a nucleic acid molecule and not present in homologous reference genomic data or sequence. Genomic data may be a published human genome, multiple published human genomes, one or more sequenced human genomes that are not published, genomic sequences obtained from the same human or subject from which the fragment is obtained, a gene or mutation database or atlas, a cancer genome atlas, or any other genomic data that may serve as a reference for comparison. Homologous means the same relative position and literally means positions in genomic sequence that share common ancestry. Thus, for example, the first 50 bases (to make up one illustrative example) of an STXBP1 gene sequenced from a tumor sample from a human are homologous to the first 50 bases of an STXBP1 gene sequenced from non-tumor cells from the same person and are homologous to the first 50 bases of an STXBP1 gene in the published human genome known as hg38 and are homologous to the first 50 bases of an STXBP1 gene sequence published in GenBank under accession number NC_000009.12. Variant means a position in one genetic sequence that does not match the homologous position in the reference genomic data or sequence. A variant may be a polymorphism or small indel. In preferred embodiments, a variant is a single nucleotide polymorphism, or SNP.

The discovery of a phased variant involves the use of a sample of known origin. Preferred embodiments involve the discovery and later detection of tumor-specific phased variants, a set of two or more phased variants that are specific to the genetic material of a tumor. Tumor-specific phased variants may be discovered by obtaining and analyzing tumor DNA. Tumor DNA may be obtained by extracting DNA from a tumor biopsy sample or a tumor specimen such as a slice (sometimes made using a microtome) that may be preserved by fixation with formalin and embedded in paraffin, to yield a formalin-fixed, paraffin embedded (FFPE) tumor or tissue slice. Extraction of DNA from FFPE may be performed using known protocols and kits. For example, one may use the FFPE tissue kit sold under the trademark QIAAMP by Qiagen GmbH (Hilden, DE).

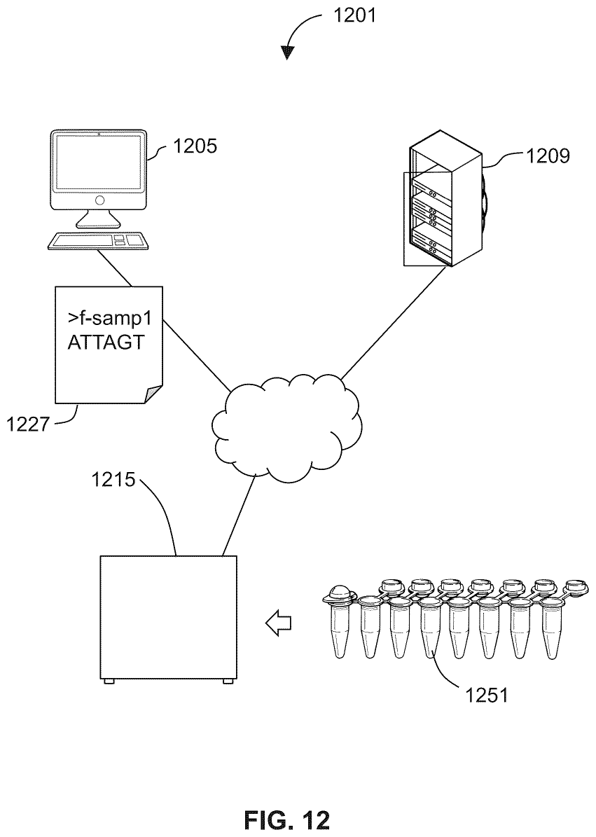

FIG. 12 shows elements of a system 1201 useful for performing methods of the invention. Nucleic acid fragment, e.g., a sample 1251, may be collected and provided in a suitable container (such as a microcentrifuge tube, test tube, tube, multiwell plate, etc.) and shipped to a lab. Methods of the invention may include obtaining the sample 1251 by receiving the shipped sample at a laboratory (e.g., tubes on dry ice) such as a clinical services laboratory. Instruments 1215 in the laboratory may be used to perform the recite steps. Preferably the instruments 1215 include a nucleic acid sequencing instrument such as a sequencing instrument sold under the trademark HISEQ or MISEQ by Illumina or a sequencing instrument from Roche, IonTorrent, Oxford Nanopore, PacBio, or Ultima Genomics. The sequencing instrument generates sequence data 1227 that is analyzed to discover or detect phased variants. The analysis is preferably performed by software operating on a computer 1205 and/or a computer system 1209. In certain embodiments, the computer 1205 is operated by a user to initiate the analysis and software packages that may be on the computer 1205 or on a remote, server or cloud, computer system 1209 may actually execute the software. The analysis may include cleaning or trimming sequence reads. Sequence reads may be assembled, e.g., by de novo assembly, mapping to a reference, or some combination thereof. The assembled sequence reads may be compared to (“mapped to”) a reference to find variants (positions where a sequence read is homologous to, but does not match, the reference). Those analytical steps may be performed by known software packages such as the Genome Analysis Tool Kit (GATK) a software package for variant discovery in high-throughput sequencing data. Variant calling as performed by computer 1205 and/or computer system 1209 may use techniques or steps described in Van der Auwera, 2020, Genomics in the Cloud: Using Docker, GATK, and WDL in Terra (1st Edition), O'Reilly Media; Poplin, 2017, Scaling accurate genetic variant discovery to tens of thousands of samples, bioRxiv, 201178; Van der Auwera, 2013, From FastQ Data to High-Confidence Variant Calls: The Genome Analysis Toolkit Best Practices Pipeline, Curr Protoc Bioinformatics, 43:11.10.1-11.10.33; DePristo, 2011, A framework for variation discovery and genotyping using next-generation DNA sequencing data, Nat Genet, 43:491-498; and McKenna, 2010, The Genome Analysis Toolkit: a MapReduce framework for analyzing next-generation DNA sequencing data, Genome Res, 20:1297-303, the contents of which are all incorporated by reference. Sequence analysis may include or involve any of the alignment, assembly, read-mapping, variant calling, or other steps described in U.S. Pat. No. 8,209,130, incorporated by reference. For example, phased variants may be identified by aligning tumor sequence reads to matched normal sequence reads to identify differences, or by aligning tumor sequence reads to a published human genome to identify differences.

Other features and embodiments are within the scope of the disclosure and may be used for any of the methods described herein. Certain embodiments of methods of the invention make use of phased-variant-specific LTC. Phased variant specific LTC is a version of LTC useful to enrich for specific phased variants, not just loci (e.g. the probes are designed to match the variant and enrich for it, potentially obviating the need for e.g. UMIs).

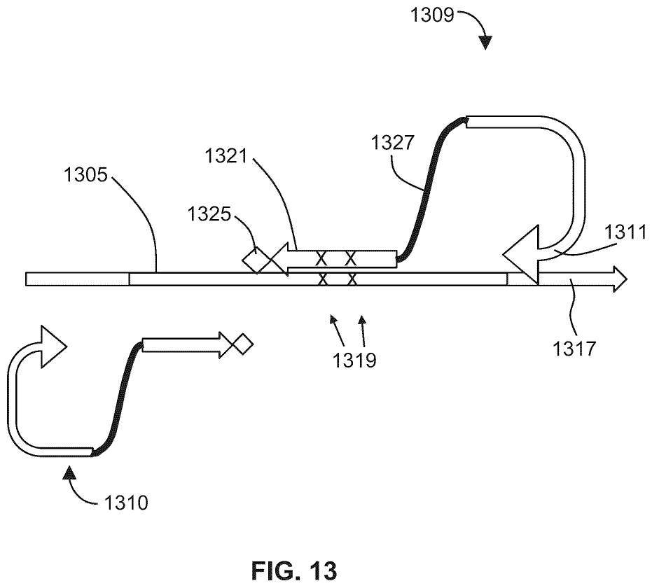

FIG. 13 shows a phase variant specific probe-dependent primer (pvPDP) 1309 according to certain embodiments. The pvPDP 1309 includes a universal primer 1311 linked at its 5′ end through a linker 1327 to a phased variant specific probe 1321 that includes a sequence that requires a fragment 1305 to include a set of phased variants 1319. The pvPDP 1209 is designed to target only particular phased variants 1319, by making the probe 1321 a perfect match to the desired target phased variants 1319. Many mutations may be targeted simultaneously in the same reaction. Additionally, probe modifications may be made to increase specificity for a given mutant or allele. For example, Locked Nucleic Acids (LNAs) may be used at a mutant or other position to increase specificity for a mutant. By designing mutant-specific probes, linked target capture can be utilized to enrich for mutant DNA only, rejecting both off target and wild-type DNA and dramatically reducing sequencing cost. As shown, the probe 1321 includes a 3′ blocker 1325 (such as an inverted base) to prevent extension. The pvPDP 1309 may be part of a set that includes a paired PDP 1310 so that amplification of the fragment 1305 is exponential (the paired PDP 1310 will anneal to the other, not pictured, strand). The fragment 1305 as shown has been ligated with adaptors 1317 that include universal primer binding sites.

In certain applications it is desirable to capture only a mutant or a particular allele sequence, such as when detecting minimal residual disease from a known tumor sequence. Mutations from an excised tumor can be used to track the presence of any disease recurrence, such as described by Gydush, 2022, Massively parallel enrichment of low-frequency alleles enables duplex sequencing at low depth, Nat Biomed Eng 6(3):257-266, incorporated by reference. By targeting only particular phased variants, off target DNA is rejected for sequencing, reducing assay cost significantly.

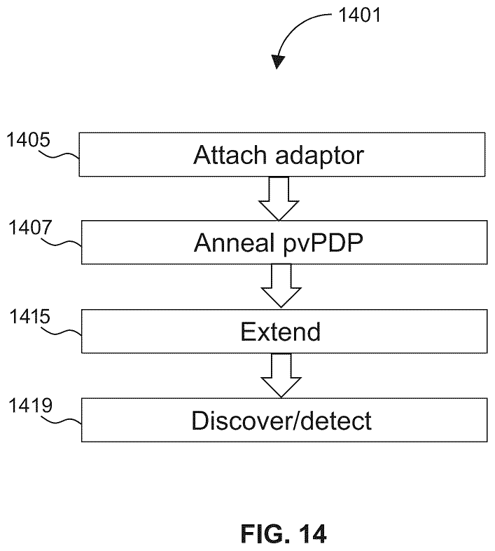

FIG. 14 shows steps of a method 1401 for targeted capture of nucleic acids using variant-specific LTC. The method 1401 includes attaching 1405 universal priming sites to a plurality of nucleic acid fragments obtained from a sample in which at least one fragment contains a phased variant; annealing 1407 a pvPDP 1309 to the nucleic acid fragments; and extending 1415 the universal primer to selectively amplify the captured phased variant 1319 without substantially amplifying any other fragment(s)p. The method 1401 preferably includes analyzing sequence data to detect 1419 the phased variant. The sample may be a biological sample from a subject and the mutant allele is associated with a tumor. In some embodiments, the target probe comprises a locked nucleic acid. Preferably the target probe is complementary to a mutant sequence comprising a phased variant and the locked nucleic acid is located at the position of the target probe corresponding to a SNP of the phased variant.

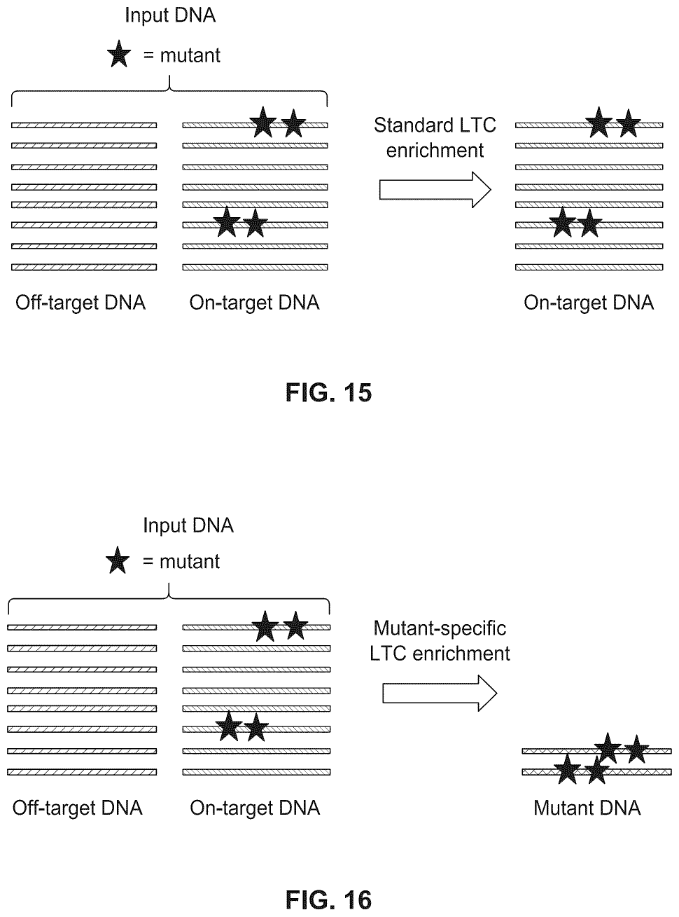

FIG. 15 summarizes information flow in LTC. The diagram shows the result of performing the method 101.

FIG. 16 illustrates information flow in phased variant specific LTC. As can be seen, the only output from the enrichment step when using phased variant specific LTC is amplicons or copies that include the phased variants.

Linked target capture probes can include modifications to improve their performance. For example, LNAs can be used to target specific mutants, or increase the melting temperature for a given probe. Intentional mismatches may also be introduced into probes, to reduce the melting temperature of a given sequence, or to reduce the capture rate of undesired sequences. Universal bases may be included, for example to minimize the impact of a possible mutation at a particular position in the target sequence.

Other features and embodiments are within the scope of the disclosure and may be used for any of the methods described herein. Certain embodiments of methods of the invention may use techniques that may be described as linked ligation. Linked ligation techniques may be used in methods for discovery of phased variants (e.g., from tumor samples) or detection of discovered phased variants in samples such as liquid biopsy samples.

The invention provides linked ligation adapters and methods allowing for increased ligation yields and simplified workflows in many capture and sequencing techniques.

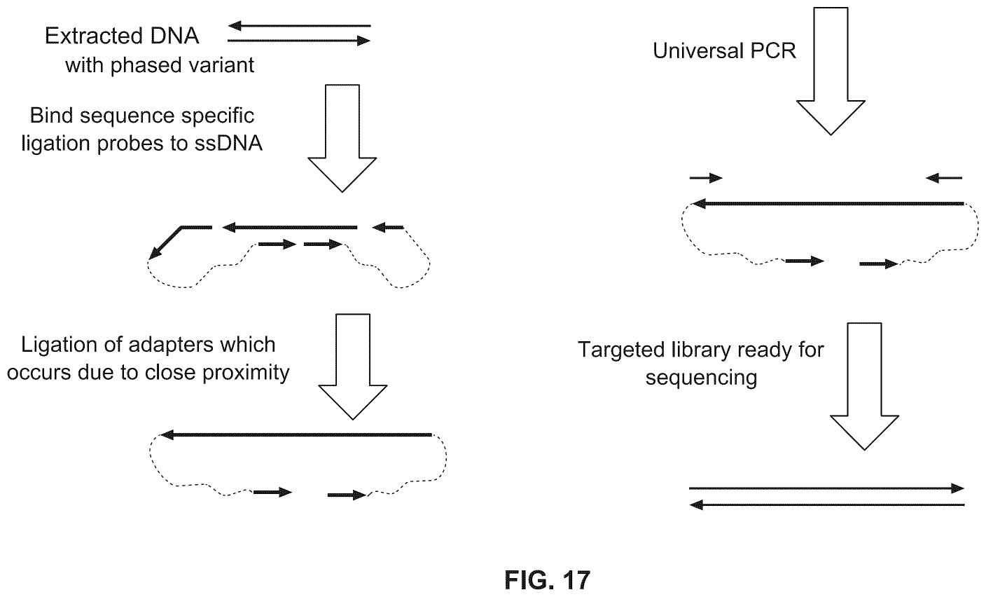

FIG. 17 shows an exemplary use of linked ligation adapters of the invention. Linked ligation adapters include adapters that may be sequencing adapters or comprise universal priming sites and are linked to target sequence specific probes, optionally phased variant specific probes. The probes are complementary to at least a portion of the target template ssDNA. The probes bind the template ssDNA strand, bringing their linked adapter into close proximity to the template and allowing for ligation of the adapters to the ends of the ssDNA template. The universal priming sites in the ligated adapters then allow for PCR amplification of the target template using universal PCR without amplifying off target nucleic acids. This results in a targeted library including sequencing adapters and ready for sequencing.

The skilled artisan will recognized that the linked ligation adaptor (LLA) workflow, as shown, is applicable to any of the LTC capture workflows discussed herein but wherein LLA uses a probe-dependent adaptor (PDA) instead of the PDP used by LTC. In LLA, the adaptor that is linked to the probe will only be ligated to the fragment when the probe anneals to its target in the fragment. The adaptor may include a universal priming site (or a sequencing instrument site) and is preferably attached to the sequence-specific probe via a linker.

By linking sequencing or universal priming site adapters to sequence specific probes, target sequence selection and capture can be combined with adapter ligation to reduce steps and increase target selectivity. Target specific probes bring adapters linked thereto into close proximity to the target sequence at which point the linked adapters may be ligated to the target sequence as shown in the drawing (arrowheads indicate the 3′ ends of nucleic acid strands). Because adapters are selectively ligated to the target sequence, subsequent amplification with universal primers complementary to sites in the ligated adapters will only amplify the target sequence, preparing a targeted library ready for sequencing. Linked ligation techniques may be used to capture nucleic acid fusions where only one side of the breakpoint is known. By linking the adapters to sequence specific probes complementary to the known portion of the fusion, methods may still be used to selectively ligate adapters and amplify only the target fusion nucleic acid for sequencing. In certain embodiments, one of the linked ligation adapter and probe molecules may be bound to a flow cell such that target nucleic acids may be captured and prepared for flow cell amplification or sequencing through adapter ligation at the same time, simplifying existing workflows. The PDA used in LLA may be phased-variant specific (similar to the situation for the pvPDP 1309) or may simply be target specific.

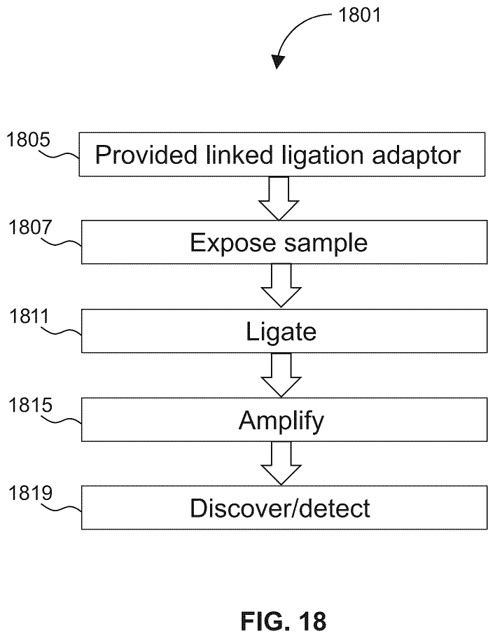

FIG. 18 shows steps of an LLA method 1801 using linked ligation adaptors to discover or detect a phased variant biomarker. The LLA method includes providing 1805 a first linked ligation adapter comprising a probe complementary to a first portion of a target nucleic acid that includes phased variants, the probe linked to a first adapter comprising a first universal priming site; exposing 1807 a sample comprising the target nucleic acid to the first linked ligation adapter; ligating 1811 the target nucleic acid to the first linked adapter; and amplifying 1815 the ligated target nucleic acid by PCR using a first universal primer complementary to the first universal priming site. The method 1801 preferably includes providing a second linked ligation adapter comprising a second probe complementary to a second portion of the target nucleic acid (e.g., the reverse PDA shown in FIG. 17), the second probe linked to a second adapter comprising a second universal priming site; exposing the sample to the second linked ligation adapter; and ligating the target nucleic acid to the second linked adapter, wherein the ligated target nucleic acid is amplified using the first universal primer and a second universal primer complementary to the second universal priming site. The method 1801 may include sequencing the target nucleic acid to obtain sequence data. The sequence data are analyzed to discover or detect 1819 the phased variants.

Preferably the sample is simultaneously exposed to the first and second linked ligation adapters. Linked ligation adapters of the invention may be used for target capture and selective amplification of target templates. Linked ligation adapters may be used with single stranded DNA (ssDNA) or, in certain embodiments, may be used with double stranded DNA (dsDNA).

By the methods described, the invention uses (i) proximity sequencing (Pro-Seq), in which a fragment is copied redundantly in a manner such that an error introduced during copying does not appear in all copies, and/or (ii) linked target capture (LTC), whereby a nucleic acid is amplified by primers that are linked to target-specific probes to discover and/or detect phased variants as biomarkers.

Specific embodiments are further illustrated and discussed.

With reference to FIG. 5, embodiment B, and FIG. 8, a sample may be assayed to discover a phased variant that is specific to a tumor. Inherent to Pro-Seq is that true phased variants from the tumor genome will reliably be found across all sequence reads from the sample, while an artefact such as a polymerase error will only appear in a measurable proportion of the sequence reads and may be identified as an artefact. Thus the disclosure provides methods 501 that use Pro-Seq with linking adapters 807 for phased variant discovery. A sample may be assayed to discover a phased variant that is specific to the tumor. In a method of using proximity sequencing for discovering a phased variant biomarker, a fragment 801 of template DNA is introduced to adaptors 805 which include a first partially double-stranded adaptor called a linking adapter 807 and second partially double stranded adaptor 808. The fragment is ligated to (i) a first adaptor 807 in which a first oligo 811 is partially annealed to a second oligo 812 and linked (via a linker such as a solid support or a molecular linker) to a primer 815 that is annealed to the second oligo 712, and (ii) a second adaptor 808 comprising a third oligo partially annealed to a fourth oligo. The first oligo 811 and the third oligo (“bottom” of adaptor 808) are attached to ends of a first strand 803 of the fragment 801. The second oligo 812 and the fourth oligo (“top” of adaptor 808) are attached to ends of a second strand 804 of the fragment 801. The phased variant biomarker with linking adapter Pro-Seq method includes extending the primer 815 to copy the second strand 804 of the fragment 701 to produce a complex 831 that comprises the first strand 803 of the fragment linked to a copy of the second strand 804 of the fragment. The second strand 804 is complementary or antisense to the first strand 803 of the fragment but the copy of the second strand 804 has the same sequence as the first strand 803 in the same 5′ to 3′ orientation. The complex 831 preferably includes the first strand of the fragment and the copy of the second strand of the fragment linked together at their 5′ ends.

Methods may include capturing the complex with a plurality of primers 905 linked to a solid support 909 (see e.g., FIG. 9) and extending the primers to form a cluster attached to the solid support. Methods may include performing at least one sequencing reaction on the cluster to generate sequence data of the fragment. Methods may include comparing the sequence data to reference data to identify at least a first variant and a second variant, relative to the reference data, present on the fragment as phased variants as a biomarker for the fragment.

For biomarker discovery, the fragment is preferably from a tumor and the reference data comprises non-tumor genomic reference data and the discovered phased variant is a biomarker specific for the tumor. The non-tumor reference may be one or more published human genomes or matched normal sequences, e.g., obtained by sequencing non-tumor DNA from the same subject.

In some embodiments, the first strand 803 and the second strand 804 (or copies thereof) are sequenced separately. The first oligo 811 and the second oligo 812 may provide the complex 831 with respective, distinct sequencing primer binding site and methods may include preforming a first sequencing reaction to determine a first sequence of the first strand and a second sequencing reaction to determine a second sequence of the sequence strand.

Methods may include sequencing the cluster to identify at least one potential phased variant (e.g., two variants on one fragment), relative to a reference, and: calling the potential phased variant as a true variant when the potential phased variant appears in sequence data from the first strand 803 and the second strand 804 of the fragment 801, or calling the potential variant as an artefact when the potential variant appears among only one of the sequence data from the first strand and the second strand.

Methods may include obtaining the fragment 801 as one of a plurality of fragments from a tumor sample (such as a formalin-fixed, paraffin embedded tumor sample, a tumor biopsy, or fresh frozen tumor DNA) and ligating adaptors that include the adaptors 805 to the plurality of fragments. Methods may include subsequently, after a subject has undergone treatment to ablate to the tumor, conducting an assay to detect the phased variant biomarker in a sample from the subject as evidence of minimal residual disease (MRD) in the subject.

Pro-Seq with linking adapters may be used for phased variant detection. Once phased variant biomarkers that are specific to a tumor have been discovered, methods of the invention are suited for the detection of such a biomarker, after it has been discovered, later in a patient's journey.

For example, after a patient has been treated to remove a tumor, it may be clinically important to later detect any evidence of the tumor in the patient. That is, it may be important to detect any minimal residual disease (MRD)

Methods of using proximity sequencing to detect MRD may include attaching adaptors 805 to a nucleic acid fragment 801 from a sample that was obtained from a subject who has been treated for a tumor. The adaptors include (i) a first linking adaptor 807 in which a first oligo is partially annealed to a second oligo and linked to a primer that is annealed to the second oligo, and (ii) a second adaptor 808 comprising a third oligo partially annealed to a fourth oligo. The first oligo and the third oligo are attached to ends of a first strand of the fragment and the second oligo and the fourth oligo are attached to ends of a second strand of the fragment. Methods include extending the primer to copy the second strand of the fragment to produce a complex that comprises the first strand of the fragment linked to a copy of the second strand of the fragment; and conducting a sequencing reaction using the complex as a template input to detect the presence of a phased variant biomarker that was previously shown to be specific to nucleic acid of the tumor. Here, Pro-Seq with linking adapters is used for phased variant detection. Methods may include capturing the complex with a plurality of copies of a primer linked to a solid substrate and extending the copies of the primer to form a cluster linked to the solid substrate. Sequencing reactions herein may include synthesizing copies of a cluster using fluorescently labeled bases and imaging the copies to detect base incorporation. Sequencing reactions herein may include a first sequencing reaction with sequencing primers specific to a first strand and a second sequencing reaction with second sequencing primers specific to copies of a second strand whereby sequence data of the first strand and the second strand are separately both obtained. Methods may include reporting that that the phased variant biomarker is present in the nucleic acid fragment when the phased variant biomarker is detected in both the sequence data of the first strand and of the second strand. Copies derived from a first strand may be attached to a first barcode and copies derived from the copy of the second strand may be attached to a second barcode (e.g., provided by the adaptors 805). Methods may include analyzing the sequence reads to confirm the presence of the first barcode and the second barcode to confirm that the copies derived from a first strand and the copies derived from a second strand were both sequenced.

Embodiments that used Pro-Seq with linking adapters may include isolating a fragment into a reaction volume (e.g., a droplet or well) prior to the attaching step. Isolating may include diluting the sample so that the nucleic acid fragment is isolated in the reaction volume.

In Pro-Seq with a linking adaptor 807, the first strand 803 of the fragment 801 and the copy of the second strand 804 of the fragment have substantially the same sequence in the same 5′ to 3′ orientation.

1-15. (canceled)

16. A method of using proximity sequencing to detect MRD, the method comprising:
isolating a nucleic acid fragment from a sample from a subject in a reaction volume with a first primer and a second primer that is linked to the first primer;
extending the first primer and the second primer to produce a complex comprising first and second copies of the fragment linked together at their 5′ ends wherein the first copy of the fragment and the second copy of the fragment have substantially the same sequence;
conducting an amplification reaction with the complex to create a plurality of linked amplicons copied from the first copy of the fragment and the second copy of the fragment;
sequencing the cluster to generate sequence reads;
assigning a base identity to each position in the fragment for which the sequence reads are in consensus thereby providing a base sequence of the fragment; and
analyzing the base sequence of the fragment to detect first and second variants that were found to be specific to a tumor of the subject when found present together on one strand of nucleic acid from the tumor.

17. The method of claim 16, wherein the first copy of the fragment is synthesized by a polymerase copying a first strand of the fragment and the second copy of the fragment is synthesized by a polymerase copying a copy of a second strand of the fragment, complementary to the first strand.

18. The method of claim 16, wherein the first primer is linked to the second primer by both being linked to a bead, the bead further comprising a plurality of primers and the method includes extending the plurality of primers to create the plurality of linked amplicons all linked to the bead.

19. The method of claim 18, wherein the amplicons linked to the bead include a first plurality of copies derived from a first strand of the fragment and a second plurality of copies derived from a second strand of the fragment.

20. The method of claim 19, wherein the copies derived from a first strand and the copies derived from a second strand are both present in the amplicons.

21. The method of claim 19, wherein the copies derived from a first strand are attached to a first barcode and the copies derived from a second strand are attached to a second barcode.

22. The method of claim 21, further comprising analyzing the sequence reads to confirm the presence of the first barcode and the second barcode to confirm that the copies derived from a first strand and the copies derived from a second strand were both sequenced.

23. The method of claim 16, wherein the reaction volume is a droplet or well.

24. The method of claim 16, wherein the isolating step comprises diluting the sample so that the nucleic acid fragment is isolated in the reaction volume.

25. The method of claim 16, further comprising, prior to performing the recited steps, identifying the first and second variants as a phased variant biomarker by a proximity sequencing technique that includes:
exposing tumor nucleic acid from a tumor sample from the subject to a first primer that (i) anneals to the tumor nucleic acid, and (ii) is linked to a second primer than anneals to the tumor nucleic acid;
extending the first primer and the second primer to produce a complex comprising linked first and second copies of the tumor nucleic acid;
generating sequences from the linked first and second copies; and
comparing the sequences to non-tumor genomic data to identify the first and second variants as a tumor-specific, phased variant biomarker.

26. The method of claim 25, wherein the phased variant biomarker is identified prior to the subject being treated to remove the tumor and the method to detect MRD is performed after the subject is treated.

27. The method of claim 16, further comprising, prior to performing the recited steps, identifying the first and second variants as a phased variant biomarker by a linked target capture technique that includes:
ligating an adaptor comprises a universal priming site to tumor nucleic acid from a tumor sample from the subject to produce an adaptor-ligated fragment;
capturing the adaptor-ligated fragment with a universal primer linked to a probe by hybridizing the probe to a site within the tumor nucleic acid and hybridizing the universal primer to the universal priming site;
extending the universal primer with a strand-displacing polymerase to make a copy of the fragment; and
sequencing the copy to identify, compared to non-tumor genomic data, the first and second variants as a tumor-specific, phased variant biomarker.

28. The method of claim 27, wherein the tumor sample comprises a formalin-fixed, paraffin embedded (FFPE) tissue slice and method includes liberating the tumor nucleic acid from the FFPE tissue slice.

29. The method of claim 27, wherein the first and second variants are identified as a tumor-specific, phased variant biomarker when:
the first variant is found in a fragment of the tumor nucleic acid but not found in a first corresponding position in non-tumor reference genomic information;
the second variant is found in the fragment but not found in a second corresponding position in the non-tumor reference genomic information; and
the first variant is found within less than about two hundred bases from the second variant in the fragment.

30. The method of claim 16, further wherein the sequencing is performed on the cluster while the cluster is attached to a bead.

31-81. (canceled)
